(19) United States
(12) Patent Application Publication
(10) Pub. No.: US 2023/0113539 A1
(43) Pub. Date: Apr. 13, 2023

(54) Traditional Chinese medicine for dispersing lung qi and detoxicating

(71) Applicant: SHANDONG BUCHANG PHARMACEUTICALS CO., LTD., Heze, Shandong (CN)

(72) Inventors: Boli ZHANG, Tianjin (CN); Qingquan LIU, Tianjin (CN); Xiumei GAO, Tianjin (CN); Junhua ZHANG, Tianjin (CN); Xinbo SONG, Tianjin (CN); Lei ZHANG, Tianjin (CN); Yi WANG, Tianjin (CN); Fengwen YANG, Tianjin (CN); Wenke ZHENG, Tianjin (CN); Tao WANG, Tianjin (CN); Yuefei WANG, Tianjin (CN); Han ZHANG, Tianjin (CN); Yuhong HUANG, Tianjin (CN); Ping WANG, Tianjin (CN); Erwei LIU, Tianjin (CN); Dailin LIU, Tianjin (CN); Jingze ZHANG, Tianjin (CN); Ming HUANG, Tianjin (CN)

(21) Appl. No.: 17/914,390

(22) PCT Filed: Mar. 17, 2021
(86) PCT No.: PCT/CN2021/081339

§ 371 Date: Sep. 26, 2022

(30) Foreign Application Priority Data
Apr. 7, 2020 (CN) 202210265774.2
Aug. 18, 2020 (CN) 202010832609.0
Aug. 18, 2020 (CN) 202010832621.1
Aug. 18, 2020 (CN) 202010833756.X
Nov. 13, 2020 (CN) 202011269872.X

(51) Int. Cl. A61K 36/17; A61K 36/704; A61K 36/736; A61K 36/85; A61K 36/8994; A61K 36/484; A61K 36/282; A61P 11/00

(52) U.S. Cl. CPC A61K 36/17; A61K 36/704; A61K 36/736; A61K 36/85; A61K 36/8994; A61K 36/484; A61K 36/282; A61P 11/00

(57) ABSTRACT

Provided are a traditional Chinese medicine for dispersing lung qi and detoxicating, a preparation method therefor, use thereof, and an analysis method. The traditional Chinese medicine contains a traditional Chinese medicine active extract. The active extract is composed of a first extract and, a second extract, herein the first extract is a water extract or an alcohol-water extract of ephedra, bitter apricot seed, raw gypsum, coix seed, rhizome of swordlike atractylodes, patchouli, Polygonum cuspidatum, verbena, reed rhizome, lepidium seed, pummelo peel, and artemisia annua, the second extract is a water extract or an alcohol-water extract of licorice, and the first extract and the second extract are separately obtained. This traditional Chinese medicine may be used for treating coronavirus diseases, such as novel coronavirus pneumonia.

### CROSS-REFERENCE TO RELATED PATENT APPLICATIONS

The present application is a National Stage of International Patent Application No. PCT/CN2021/081339, filed on Mar. 17, 2021, and claims the priority of Chinese Patent Application No. CN202010265774.2, filed on Apr. 7, 2020, CN202010832621.1, filed on Aug. 18, 2020, CN202010833756.X, filed on Aug. 18, 2020, CN202010832609.0, filed on Aug. 18, 2020, CN202011269872.X, filed on Nov. 13, 2020, the disclosures of which are incorporated by reference in their entirety.

### TECHNICAL FIELD

The present invention belongs to the field of traditional Chinese medicine, and in particular relates to a traditional Chinese medicine for dispersing lung qi and detoxicating, the traditional Chinese medicine may disperse lung qi and detoxicate, treat diseases caused by coronaviruses, and treat novel coronavirus pneumonia (COVID-19). The present invention further relates to a preparation method for the traditional Chinese medicine for dispersing lung qi and detoxicating, and a method for preparing a solid preparation of the traditional Chinese medicine.

### BACKGROUND

Coronaviridae viruses are a large class of viruses that exist widely in nature, infecting vertebrates, such as humans, mice, pigs, cats, dogs, wolves, chickens, cattle, and birds. A novel coronavirus (SARS-CoV-2) belongs to a β-genus coronavirus in the Coronaviridae, and genetic characteristics are significantly different from those of SARSr-CoV and MERSr-CoV. The virus is sensitive to ultraviolet light and heat, and the virus may be effectively inactivated at 56° C. for 30 minutes by lipid solvents such as ether, 75% ethanol, a chlorine-containing disinfectant, a peracetic acid and chloroform. Based on existing epidemiological survey and research results, the incubation period is 1-14 days, and 3-7 days at most; the source of infection is mainly patients infected with novel coronaviruses, and asymptomatic infected persons may also become the source of infection; the main transmission routes are respiratory droplet and contact transmission, and there is a possibility of transmission by aerosols while exposed to high concentrations of the aerosols for a long time in a relatively closed environment. Other transmission routes are yet to be identified; and the population is generally susceptible.

For the novel coronavirus, most of drugs and methods used in the clinical treatment of Western medicine are derived from the experience of treating SARS and H1N1 influenza, such as antiviral and anti-infective drugs, a gamma globulin, methylprednisolone, an immunotherapy using tocilizumab, as well as convalescent plasma and mechanically assisted ventilation treatment, and a specific therapeutic drug and a treatment scheme are not yet found. Traditional Chinese medicine has a long history in treatment of plague, and plays a great role and remarkable effect in treatment process of infectious diseases such as SARS and H1N1 influenza. Pneumonia caused by the novel coronavirus infection is a result of a game between the virus infection and the body immune state in vivo. The traditional Chinese medicine may play a role in the whole course of the treatment and in all directions, and it focuses on mobilizing the own disease resistance of a body, improving clinical symptoms, reducing complications and improve quality of life.

According to a theory of the traditional Chinese medicine, novel coronavirus pneumonia (COVID-19) belongs to the category of “epidemic” diseases in the traditional Chinese medicine. A pathogenic factor is dampness toxin, a core pathogenesis is dampness toxin, lung obstruction and qi suppression, and pathological characteristics may be summarized as “dampness, toxin, close, and empty”. Results of autopsy and puncture histological observation of patients with the novel coronavirus pneumonia show that lungs are consolidated to varying degrees, the number of spleen and lymphocytes is significantly reduced and decreased, a part of vascular endothelium is shed, endocarditis and thrombosis are caused, gallbladders are highly filled, kidney interstitial congestion and glomerulus renal tubule lesion are caused, it is indicated that the novel coronavirus pneumonia causes more serious lung, kidney and immune system damage (Wang Yi, Li Xiang, Zhang Junhua, Xue Rui, Qian Jingyang, Zhang Xiaohui, Zhang Han, Liu Qingquan, Fan Xiaohui, and Zhang Boli. Study on mechanism of dispersing lung qi and detoxicating decoction in treatment of novel coronavirus pneumonia based on network pharmacology. China Journal of Traditional Chinese Medicine, 1-9, 2020).

The traditional Chinese medicine has a long history in the treatment of viral diseases and accumulates rich experience. The traditional Chinese medicine also plays an important role in the control of many modern epidemic diseases. For example, in an aspect of anti-influenza viruses, a lot of researches are done on Chinese patent medicines. The researches show that the resistance to influenza viruses by the Chinese patent medicines mainly includes two points: by killing the virus, and reducing the invasion of the influenza viruses on host cells, it may directly act on virus cells, and has direct antiviral drugs including conventional heat-clearing and detoxifying drugs such as isatis root and honeysuckle; the body inflammatory response is adjusted by various mechanisms, to enhance the body immunity, improve its clinical symptoms, and prevent diseases from deepening. Drugs with indirect antiviral properties include astragalus, wolfberry, codonopsis and the like. These pharmaceutical ingredients may enhance the defense function of the body. The Chinese patent medicines may also enhance an ability to resist the viruses by enhancing the body non-specific immunity and specific immunity, and the advantages are apparent. The anti-infection of the traditional Chinese medicine presents the characteristics of multi-mechanism and multi-target. Based on the principle of a traditional Chinese medicine dialectical theory, pathogens in the body are eliminated, the body immunity is improved, and it has significant effects in aspects of treatment and defense of influenza. Early intervention of the traditional Chinese medicine may block the course of the disease, improve the symptoms, and promote inflammatory absorption. The addition of the traditional Chinese medicine for promoting blood circulation and removing blood stasis and dredging collaterals may slow down the occurrence of pulmonary interstitial fibrosis during the recovery period; and the individualized treatment with the traditional Chinese medicine may effectively shorten the hospitalization time of patients, and reduce sequelae, complications and toxic side effects of Western medicines. The National Administration of Traditional Chinese Medicine brings 8 Chinese patent medicines of Qingkailing injection, Houttuynia cordata injection, Banlangen granules, Xinxue granules, Jinlian Qingre granules, Dengzhan Xixin injection, Fufang Kushen injection and Xiangdan injection into “Technical Plan for Treatment and Prevention of Severe Acute Respiratory Syndrome (SARS) with Traditional Chinese Medicine (Trial)”, and it is believed that these traditional Chinese medicines have apparent improvement effects on different pathological links of SARS.

The traditional Chinese medicine also has good effects in treatment of viral infectious diseases. For example, it achieves the remarkable results in treatment of SARS, H1N1 and other diseases. It is confirmed from researches that the overall regulation of the traditional Chinese medicine may enhance the body immunity, and at the same time inhibit and kill the viruses. It is shown from evidence that the traditional Chinese medicine plays an important role in treatment of the novel coronavirus pneumonia. The national ministries and commissions issue a notice on the use of integrated traditional Chinese and Western medicines to prevent and treat this time of COVID-19, it is proposed that the symptom features and evolution laws of the traditional Chinese medicine are grasped, and a better entry point for the traditional Chinese medicine is explored, especially it is emphasized that the attention should be paid to looking for some Chinese patent medicines and prescriptions that are really effective in clinical evaluation. At present, the combination of traditional Chinese and Western medicines is a main treatment method for COVID-19, of which the use of traditional Chinese medicine decoction accounts for 88.2%. Compared with a Western medicine treatment group, patients in an integrated traditional Chinese and Western medicine treatment group are significantly shorter in body temperature recovery time and average length of hospital stay, and are significantly reduced in accompanying symptom disappearance rate, CT image improvement rate, clinical cure rate, incidence of common-type to severe-type and critical-type and mortality rate. In addition, according to clinical surveys, 80% of the patients with severe COVID-19 are willing to accept the traditional Chinese medicine treatment, 90% of the patients with mild symptoms are willing to accept the traditional Chinese medicine intervention, and isolated patients also hope that the traditional Chinese medicine intervene early. Therefore, it is urgent to research and develop prescriptions with significant curative effects in clinical evaluation, and it is of great significance to the prevention and control of the epidemic situation.

At present, there is an urgent need to develop the traditional Chinese medicine preparations for the treatment of COVID-19 with the significant clinical efficacy, and it is specifically treated for different stages of COVID-19 in order to cover more patients.

### SUMMARY

One purpose of the present invention is to provide a prescription, a preparation method, a use and an analysis method for a traditional Chinese medicine for dispersing lung qi and detoxicating.

One purpose of the present invention is to provide a traditional Chinese medicine that may effectively treat a disease caused by a coronavirus, a preparation of the traditional Chinese medicine includes a traditional Chinese medicine prescription composition and a dosage ratio range, especially a traditional Chinese medicine solid preparation for COVID-19, and the solid preparation includes a traditional Chinese medicine active extract.

Another purpose of the present invention is to provide a method for preparing a traditional Chinese medicine preparation that may effectively treat a disease caused by a coronavirus, especially COVID-19.

According to a theory of traditional Chinese medicine, for the overall syndrome performance of COVID-19, the inventors of the present application creatively obtain the composition and ratio of the effective traditional Chinese medicine prescription by a large amount of clinical practice on the basis of ancient traditional Chinese medicine prescriptions.

The inventiveness of the present invention lies in the fact that: the inventors of the present application, Professor Zhang Boli and Professor Liu Qingquan, according to a dialectical basis of a novel coronavirus pneumonia disease, combined with their years of clinical experience, and by researches and summary, draw up an effective prescription of the traditional Chinese medicine. This prescription is based on four classic famous prescriptions, namely Maxing Shigan decoction in “Treatise on Febrile Diseases”, Maxing Yigan decoction and Tingli Dazao Xiefei decoction in “Synopsis of Golden Chamber”, and Qianjin Weijing decoction in “Medical Secretes of official”, added with medicinal materials such as pummelo peel, patchouli, artemisia annua, Polygonum cuspidatum, verbena, and atractylodes, and clinically used for treatment of common-type patients with COVID-19. In terms of the composition of medicinal flavors, it is composed of traditional Chinese medicines with four types of effects of relieving superficies syndrome with pungent and warm natured drugs, purging intense heat and detonicating, clearing heat and expelling damp, and relieving cough and asthma, the whole prescription is mainly based on dispersing lung qi, clearing heat and relieving superficies syndrome, supplemented by dispelling dampness and removing drink, and also for relieving cough and asthma. It is suitable for dampness and poison in the lung, fever, cough with little or yellow phlegm, suffocation and shortness of breath, abdominal distension, inhibited defecation, dark red tongue nature, fat tongue body, yellow greasy or yellow dry coating, slippery pulse or string slippery symptoms. Therefore, from the perspective of traditional Chinese medicine etiology, COVID-19 damages two meridians of lung and spleen in an early stage; diseases and pathogens damage the lung, resulting in fever, fatigue, body ache, and coughing, which are consistent with the exogenous symptoms of the mild-type and common-type patients; and the diseases and pathogens damage the spleen, symptoms such as anorexia, abdominal distension, nausea, vomiting, and inhibited defecation appear, which are consistent with the gastrointestinal symptoms of the mild-type and common-type patients. Dampness traps the spleen, which causes the spleen to lose its healthy function and aggravates the syndrome of stagnation of lung water dampness. Therefore, granules for dispersing lung qi and detoxicating target to the two meridians of the lung and spleen from the prescription, the prescription selects ephedra, bitter apricot seed, raw gypsum, and licorice in the Maxing Shigan decoction prescription as a monarch drug, which is pungent and cool to vent, and clears the lungs and relieves asthma; in the prescription, reed rhizome, coix seed, lepidium seed, and pummelo peel are used as an adjuvant drug, which together play the effect of clearing heat and purging the lungs, dispelling dampness and promoting water; it is supplemented with rhizome of swordlike atractylodes and patchouli, which helps dispelling dampness; artemisia annua and Polygonum cuspidatum help to clear away heat and detoxify, and may suppress and kill the novel coronaviruses according to the characteristics of the “damp and poison epidemic” of this time of COVID-19; verbena promotes blood circulation, activates collaterals and eliminates stagnation, as to help clearing lung and activating; and many drugs in this prescription are used in combination, and used for dispersing and clearing in both symptoms and root causes, which together play the effects of dispersing lung qi and resolving dampness, clearing heat and removing pathogenic factors, purging lung and detoxifying.

The function of the traditional Chinese medicine preparation of the present invention is mainly for the syndrome of dampness and stagnation of the lung, and is used for treating the common-type of COVID-19, the symptoms are fever, cough with little or yellow phlegm, suffocation and shortness of breath, abdominal distension, inhibited defecation, dark red tongue nature, fat tongue body, yellow greasy or yellow dry coating, slippery pulse or slippery string. It is clinically proved that the prescription may effectively treat the common-type patients with COVID-19. This prescription is one of important prescriptions for “three-medicine and three-prescription” for the prevention and treatment of COVID-19, and plays an important role in the prevention and treatment process of this time of COVID-19.

The prescription and dosage ratio of the traditional Chinese medicine preparation of the present invention are obtained by the inventors of the present application after a lot of explores and summaries for the clinical practice of the novel coronaviruses. The dosage of each traditional Chinese medicine prescription for treating COVID-19 has the good clinical efficacy, within the following weight part ranges.

The traditional Chinese medicine preparation of the present invention is prepared from the following raw materials by weight: 110˜190 parts of ephedra, 460˜540 parts of Polygonum cuspidatum, 330˜420 parts of bitter apricot seed, 710˜790 parts of verbena, 710˜790 parts of raw gypsum, 710˜790 parts of reed rhizome, 710˜790 parts of coix seed, 330˜420 parts of lepidium seed, 210˜290 parts of rhizome of swordlike atractylodes, 330˜420 parts of pummelo peel, 330˜420 parts of patchouli, 210˜290 parts of licorice, and 250˜350 parts of artemisia annua.

As a preferred prescription of the traditional Chinese medicine preparation of the present invention, the traditional Chinese medicine preparation is prepared from the following raw materials by weight: 130˜170 parts of ephedra, 480˜520 parts of Polygonum cuspidatum, 350˜400 parts of bitter apricot seed, 730˜770 parts of verbena, 730˜770 parts of raw gypsum, 730˜770 parts of reed rhizome, 730˜770 parts of coix seed, 350˜400 parts of lepidium seed, 230˜270 parts of rhizome of swordlike atractylodes, 350˜400 parts of pummelo peel, 350˜400 parts of patchouli, 230˜270 parts of licorice, and 270˜330 parts of artemisia annua.

As a further preferred prescription of the traditional Chinese medicine preparation of the present invention, the traditional Chinese medicine preparation is prepared from the following raw materials by weight: 150 parts of ephedra, 500 parts of Polygonum cuspidatum, 375 parts of bitter apricot seed, 750 parts of verbena, 750 parts of raw gypsum, 750 parts of reed rhizome, 750 parts of coix seed, 375 parts of lepidium seed, 250 parts of rhizome of swordlike atractylodes, 375 parts of pummelo peel, 375 parts of patchouli, 250 parts of licorice, and 300 parts of artemisia annua.

On the basis of acquiring the above clinically effective prescriptions and ratios, the present invention also combines modern science technologies. In the process of developing a new traditional Chinese medicine, on the basis of guaranteeing the safety, effectiveness, stability and controllability of a traditional Chinese medicine compound preparation, the classic famous prescriptions are fully respected, and the safety and efficacy of clinical applications are used as a goal, to rationally design a process route. Herein, a principle of the process route selection is to guarantee that a product is “safe, effective, and quality-controllable”. The present invention is based on clinical standard decoction of the first-line anti-epidemic, and combined with research methods of the classic famous prescriptions to determine an extraction and enrichment process route of effective active substance components of the traditional Chinese medicine preparation of the present invention. The influence of ephedra and licorice herbs on index components in the combined decoction process is investigated, and a single decoction process of the licorice is determined. The decoction specification of the traditional Chinese medicine is followed, and a purpose of an extraction process that the raw gypsum is decocted firstly, and followed with the patchouli and artemisia annua is to reduce the amount of the extract under the premise of guaranteeing and retaining the index components, it is beneficial to the preparation of granules that are easily accepted by the patients, and increase the compliance.

A traditional Chinese medicine formula of the present invention (sometimes referred to as a “prescription for dispersing lung qi and detoxicating” in the description) is finally determined by the above large number of the scientific researches, and it is found in clinical treatment practice that the prescription for dispersing lung qi and detoxicating has the effect on the patients with COVID-19, and shows the unique advantage especially in an aspect of reducing the conversion of mild patients to severe disease. Based on the above findings, the present inventors complete the present invention.

The present invention may be described in different aspects, and the inventions described in these aspects and any forms thereof are independent of each other but also related to each other, and constitute the content of the present invention in combination.

On the one hand, the present invention provides a traditional Chinese medicine solid preparation for treating a coronavirus disease (especially COVID-19), the traditional Chinese medicine solid preparation includes a traditional Chinese medicine active extract, including a first extract and a second extract, or consisting of the first extract and the second extract. The first extract is an extract obtained by extracting ephedra, bitter apricot seed, raw gypsum, coix seed, rhizome of swordlike atractylodes, patchouli, Polygonum cuspidatum, verbena, reed rhizome, lepidium seed, pummelo peel, and artemisia annua with water or alcohol-water solution; and the second extract is an extract obtained by extracting licorice with water or alcohol-water solution, herein the extraction of the first extract and the second extract is performed separately. The extraction or preparation process of the first extract is characterized in that the raw gypsum is decocted firstly, and after the extraction, the ephedra, bitter apricot seed, coix seed, rhizome of swordlike atractylodes, Polygonum cuspidatum, verbena, reed rhizome, lepidium seed and pummelo peel are added to extract, and it is filtered, to obtain a first filtrate, the artemisia annua and patchouli are added to a medicinal residue to extract, it is filtered, to obtain a second filtrate, and the first filtrate and the second filtrate are combined to obtain the first extract. According to the traditional Chinese medicine composition of the present invention, in addition to the active extract of the traditional Chinese medicine of the present invention, it may further contain a pharmaceutically acceptable excipient.

Herein the first water extract may be a water extract obtained by a method including the following steps.

(1) the raw gypsum is placed in water which is 3 to 15 times in amount to extract for 10 to 90 minutes, then the ephedra, bitter apricot seed, coix seed, rhizome of swordlike atractylodes, Polygonum cuspidatum, verbena, reed rhizome, lepidium seed and pummelo peel are added, and extracted for one or more times with 3 to 15 times of the water in amount, each time is 20 to 90 minutes, it is filtered, and filtrates are combined while being extracted for many times, to obtain a first filtrate, and a medicinal residue is directly used in the next step.

(2) the artemisia annua and patchouli are added to the medicinal residue, 3 to 10 times of the water in amount is added, it is extracted for one or more times, each time is 10 to 80 minutes, it is filtered, and filtrates are combined while being extracted for many times, to obtain a second filtrate.

(3) the first filtrate and the second filtrate are combined, and concentrated, to obtain the first extract.

More preferably, the first water extract may be a water extract prepared by a method including the following steps.

(1) the raw gypsum is placed in water which is 10 times in amount to extract for 20 to 40 minutes, more preferably 30 minutes, then the ephedra, bitter apricot seed, coix seed, rhizome of swordlike atractylodes, Polygonum cuspidatum, verbena, reed rhizome, lepidium seed and pummelo peel are added, and extracted for once or twice with 10 times of the water in amount, each time is 1 hour, it is filtered, and filtrates are combined while being extracted for twice, to obtain a first filtrate, and a medicinal residue is directly used in the next step.

(2) the artemisia annua and patchouli are added to the medicinal residue, 6 times of the water in amount is added, it is extracted for once or twice, each time is 20 to 40 minutes, it is filtered, and filtrates are combined while being extracted for twicewice, to obtain a second filtrate.

(3) the first filtrate and the second filtrate are combined, and concentrated, to obtain the first extract.

Herein the second water extract may be a water extract obtained by a method including the following steps.

The licorice is placed in 3 to 15 times of the water in amount to extract for one or more times, each time is 20 to 90 minutes, it is filtered, filtrates are combined while being extracted for many times, and the filtrate is concentrated, to obtain the second water extract.

The solid preparation of the traditional Chinese medicine according to the present invention may further contain, in addition to the active extract of the traditional Chinese medicine of the present invention, a pharmaceutical excipient suitable for the solid preparation.

On the other hand, the present invention provides a method for preparing the solid preparation of the traditional Chinese medicine according to the present invention, and it includes the following steps.

(1) the raw gypsum is placed in 3 to 15 times of water or alcohol-water solution in amount to extract for 10 to 90 minutes, then the ephedra, bitter apricot seed, coix seed, rhizome of swordlike atractylodes, Polygonum cuspidatum, verbena, reed rhizome, lepidium seed and pummelo peel are added, and extracted for one or more times with 3 to 15 times of the water in amount, each time is 20 to 90 minutes, it is filtered, and filtrates are combined while being extracted for many times, to obtain a first filtrate, and a medicinal residue is directly used in the next step;

(2) the artemisia annua and patchouli are added to the medicinal residue, it is extracted for one or more times with 3 to 10 times of the water in amount, each time is 10 to 80 minutes, it is filtered, filtrates are combined while be extracted for many times, to obtain a second filtrate;

(3) the first filtrate and the second filtrate are combined, and concentrated, to obtain first concentrated solution;

(4) the licorice is placed in 3 to 15 times of the water or alcohol-water solution in amount to extract for one or more times, each time is 20 to 90 minutes, it is filtered, and filtrates are combined while being extracted for many times, the filtrate is concentrated, to obtain second concentrated solution;

(5) the first concentrated solution and the second concentrated solution are combined, and further concentrated to a relative density of about 1.01-1.05 (60° C.), to obtain a final concentrate;

(6) the obtained final concentrate is spray-dried, to obtain a medicinal powder.

(7) an appropriate amount of a pharmaceutically acceptable excipient is added to the obtained medicinal powder, and the desired traditional Chinese medicine composition is prepared by a method known in the field.

In yet another aspect, the present invention provides a use of the traditional Chinese medicine active extract or the traditional Chinese medicine composition according to the present invention in preparation of a drug for treating a coronavirus disease (for example, COVID-19). Alternatively, the present invention provides a method for treating a patient with a coronavirus disease (for example, COVID-19) using the traditional Chinese medicine composition of the present invention, the method includes administering a therapeutically effective amount of the traditional Chinese medicine composition of the present invention to the patient with the coronavirus disease (for example, COVID-19). Alternatively, the present invention provides a traditional Chinese medicine composition for treating a coronavirus disease (for example, COVID-19) patient, and this traditional Chinese medicine composition contains the traditional Chinese medicine active extract of the present invention.

It is proved from results of preclinical tests and clinical treatment that the traditional Chinese medicine active extract and the traditional Chinese medicine composition of the present invention may improve the ability of the patient immune system to fight against the coronaviruses, one or more symptoms of the patient are alleviated or eliminated, and the mild symptom is not converted to the severe disease or the probability of the conversion is small, the severe disease is converted to the common-type or mild symptom or the probability of the conversion is increased, the recovery time of the patients is shortened, the course of the disease is shortened, and the nucleic acid turns to negative faster. Therefore, the traditional Chinese medicine active extract and the traditional Chinese medicine composition according to the present invention may effectively treat the patient with the coronavirus disease, such as the patient with COVID-19, especially for mild-type and common-type patients, the curative effect is remarkable, and it may significantly reduce the symptoms such as fever, cough, suffocation asthma and fatigue, CT diagnosis after treatment also shows the significant improvement in the related symptoms.

One or more embodiments provide a traditional Chinese medicine, a traditional Chinese medicine extract, a traditional Chinese medicine active extract, a traditional Chinese medicine composition, a traditional Chinese medicine prescription, a traditional Chinese medicine formula, a traditional Chinese medicine preparation, a traditional Chinese medicine compound preparation, a traditional Chinese medicine solid preparation, a traditional Chinese medicine granule, a traditional Chinese medicine capsule, a traditional Chinese medicine pill, and a traditional Chinese medicine drop pill of the present application, and it is used as a drug.

One or more embodiments of the present application provide a traditional Chinese medicine, a traditional Chinese medicine extract, a traditional Chinese medicine active extract, a traditional Chinese medicine composition, a traditional Chinese medicine prescription, a traditional Chinese medicine formula, a traditional Chinese medicine preparation, a traditional Chinese medicine compound preparation, a traditional Chinese medicine solid preparation, a traditional Chinese medicine granule, a traditional Chinese medicine capsule, a traditional Chinese medicine pill, and a traditional Chinese medicine drop pill of the present application, and it is used in a method for treating, inhibiting and/or preventing COVID-19.

One or more embodiments of the present application provide a method for treating, inhibiting and/or preventing COVID-19, including administering a traditional Chinese medicine, a traditional Chinese medicine extract, a traditional Chinese medicine active extract, a traditional Chinese medicine composition, a traditional Chinese medicine prescription, a traditional Chinese medicine formula, a traditional Chinese medicine preparation, a traditional Chinese medicine compound preparation, a traditional Chinese medicine solid preparation, a traditional Chinese medicine granule, a traditional Chinese medicine capsule, a traditional Chinese medicine pill, and a traditional Chinese medicine drop pill of the present application to a subject in need thereof.

### BRIEF DESCRIPTION OF THE DRAWINGS

Drawings described herein are used to provide further understanding of the present invention, and constitute a part of the present invention. Exemplary embodiments of the present invention and descriptions thereof are used to explain the present invention, and do not constitute improper limitation to the present invention.

FIG. 1—shows an effect of a traditional Chinese medicine granule (XFBD) according to the present invention on a lung index in a mouse.

FIGS. 2—(2A and 2B) shows an effect of a traditional Chinese medicine granule according to the present invention on a serum gastrointestinal hormone in a mouse.

FIGS. 3—(3A, 3B, 3C and 3D) shows an effect of a traditional Chinese medicine granule according to the present invention on a content of an inflammatory factor in a lung tissue of a mouse.

### DETAILED DESCRIPTION OF THE EMBODIMENTS

The present invention is already summarized above in general aspects, and the present invention is further described in detail below in combination with embodiments.

In order to accurately understand terms used in the present invention, the meanings of some terms are specially defined below. For terms that are not specifically defined herein, they have the meanings generally understood and accepted by those skilled in the art. If the meaning of a certain term defined herein is inconsistent with the meaning generally understood and accepted by those skilled in the art, the meaning of the term shall be governed by the meaning defined herein.

A term “ephedra” as used in the present invention refers to a dried herbaceous stem of Ephedra sinicaStapf, Ephedra intermedia Schrenk et C. A. Mey. or Ephedra equisetinaBge.

A term “bitter apricot seed” used in the present invention refers to a dried mature seed of Prunus armeniaca L. var. ansu Maxim., Prunus sibirica L., Prunus mandshurica (Maxim.) Koehne or Prunus armeniaca L. A mature fruit is harvested in summer.

A term “raw gypsum” used in the present invention refers to a raw gypsum in the sulfate mineral anhydrite family, mainly containing a calcium sulfate (CaSO₄.2H₂O) containing water. After excavation, miscellaneous stones and sediment are removed.

A term “coix seed” used in the present invention refers to a dried mature seed kernel of Coixlacryma-jobi L.var.ma-yuen (Roman.) Stapf.

A term “rhizome of swordlike atractylodes” used in the present invention refers to a dried rhizome of Atractylodeslancea (Thunb.) DC. or Atractylodes chinensis (DC.) Koidz., and a processed product fried with a wheat bran; and it may reduce its dryness, and the medicinal properties are mild.

A term “patchouli” used in the present invention refers to a dried aerial part of Pogostemoncablin (Blanco) Benth.

A term “Polygonum cuspidatum” used in the present invention refers to a dried rhizome and root of Polygonum cuspidatum Sieb.et Zucc.

A term “verbena” used in the present invention refers to a dried aerial part of Verbena Officinalis L.

A term “reed rhizome” used in the present invention refers to a fresh or dried rhizome of Phragmites communis Trin.

A term “lepidium seed” used in the present invention refers to a dried mature seed of Descurainiasophia (L.) Webb. ex Prantl. or Lepidium apetalum Willd.

A term “pummelo peel” used in the present invention refers to an immature or near-mature dried outer peel of Citrus grandis ‘Tomentosa’ or Citrus grandis (L.) Osbeck.

A term “artemisia annua” used in the present invention refers to a dry aerial part of Artemisia annua L.

A term “licorice” used in the present invention refers to a dried root and rhizome of Glycyrrhiza uralensis Fisch., Glycyrrhiza inflata Bat. or Glycyrrhiza glabra L.

A term “traditional Chinese medicine active extract” used in the present invention refers to a substance with anti-coronavirus activity in any forms obtained by extracting any forms of the traditional Chinese medicinal materials (including a traditional Chinese medicinal material decoction piece, and traditional Chinese medicinal material powder, such as traditional Chinese medicinal material micronized powder) with a suitable solvent such as water or water-alcohol solution, including a specific active ingredient and a mixture containing the active ingredient. Forms of the extract include but not limited to solid, semi-solid, solution, suspension, concentrate, paste-like body and powder.

The water suitable for extracting the traditional Chinese medicinal material to obtain the traditional Chinese medicine active extract of the present invention refers to various waters that may be used for preparing the traditional Chinese medicine active extract, including medicinal water, such as distilled water and deionized water.

A term “alcohol-water solution” used in the present invention refers to aqueous solution of alcohol at a suitable concentration (for example, a low concentration, especially 10-50% v/v). Examples of the suitable alcohol include lower alcohol, preferably ethanol. Under a certain condition, the aqueous solution of the alcohol with a concentration higher than 50% v/v may also be used.

A term “extracting for one or more times” used in the present invention refers to generally extracting for 1 to 3 times, preferably 1 or 2 times, and in the special case, it may be extracted for more times as required.

Terms “patient” and “subject” used in the present invention may be used interchangeably, and refer to mammals, especially a human, who are susceptible to the coronavirus diseases or are susceptible to the coronaviruses.

A term “coronavirus” used in the present invention refers to a virus belonging to Coronaviridae, particularly to a virus of the Coronavirus genus, and more particularly to SARS-CoV-2, including any mutants of them.

A term “coronavirus disease” used in the present invention refers to a disease caused by the virus of Coronaviridae, particularly the virus of the Coronavirus genus, and more particularly SARS-CoV-2, including any mutants of them.

A term “treatment” used in the present invention means improving the ability of the immune system in the patient to fight against the coronavirus, alleviating or eliminating one or more symptoms of the coronavirus disease in the patient, preventing the conversion from mild-type to severe disease, improving the probability of the conversion from a critical patient to an ordinary patient, shortening the recovery time of the patient, shortening the course of the disease, and promoting the nucleic acid to turn to negative.

A phrase “therapeutically effective amount” used in the present invention refers to an amount of the traditional Chinese medicine active extract or the pharmaceutical composition according to the present invention which is provided for an expected clinical therapeutic effect while being administered to the patient, and the clinical effect is that the ability of the patient immune system to fight against the coronavirus is improved, one or more symptoms of the patient are alleviated or eliminated, the mild-type disease is not converted into the severe disease, the probability of the conversion from the severe patient to the common-type or mild-type patient is increased, the recovery time of the patient is shortened, the course of the disease is shortened, and the nucleic acid turns to negative faster.

A term “pharmaceutically acceptable excipient” used in the present invention means any excipients conventionally used in the field of pharmaceutical preparations, as long as the excipient does not produce the adverse effect or influence on the expected quality and curative effect of the traditional Chinese medicine composition of the present invention. For example, the pharmaceutically acceptable excipients include a diluent, a carrier, a filler, a binder, a wetting agent, a disintegrant, an absorption enhancer, a surfactant, an adsorption carrier and a lubricant and the like conventionally used in the pharmaceutical field. The commonly used diluents are sucrose, dextrin, starch, lactose, mannitol, xylitol, bifidose and the like. The commonly used wetting agents are mainly water, different concentrations of ethanol and the like. The commonly used binders include a polymer binder, of which there are many types, such as ethyl cellulose, polyvinyl pyrrolidone, sodium carboxymethyl cellulose, polyethylene glycol, and sodium alginate. The commonly used disintegrants include microcrystalline cellulose, sodium carboxymethyl starch and the like. Those skilled in the art may select and determine the suitable excipients in the traditional Chinese medicine composition of the present invention according to the content disclosed in the description. The choice of a particular excipient may depend on a mode of administration used to treat the particular patient or disease type and state. If necessary, a flavoring agent, a preservative, a sweetener and the like may also be added to the pharmaceutical composition. Preparation methods for the suitable pharmaceutical compositions for a particular mode of administration are well-known within a knowledge scope of those skilled in the art.

All numerical ranges disclosed in the present application are inclusive of an end value thereof, and include any sub-ranges within the range which is not expressly recited.

According to one aspect of the present invention, the present invention relates to a traditional Chinese medicine composition or a traditional Chinese medicine preparation that may be used to treat a coronavirus disease (for example, COVID-19), the traditional Chinese medicine composition or the traditional Chinese medicine preparation includes ephedra, bitter apricot seed, raw gypsum, coix seed, rhizome of swordlike atractylodes, patchouli, Polygonum cuspidatum, verbena, reed rhizome, lepidium seed, pummelo peel, artemisia annua and licorice, or may also be composed or prepared by these traditional Chinese medicinal materials. For example, the traditional Chinese medicine composition or the traditional Chinese medicine preparation may contain 6 parts of ephedra, 15 parts of bitter apricot seed, 30 parts of raw gypsum, 30 parts of coix seed, 10 parts of rhizome of swordlike atractylodes, 15 parts of patchouli, 20 parts of Polygonum cuspidatum, 30 parts of verbena, 30 parts of reed rhizome, 15 parts of lepidium seed, 15 parts of pummelo peel, 12 parts of artemisia annua and 10 parts of licorice, or composed or prepared by these traditional Chinese medicinal materials. Preferably, the traditional Chinese medicine composition or the traditional Chinese medicine preparation contains 150 g of ephedra, 375 g of bitter apricot seed, 750 g of raw gypsum, 750 g of coix seed, 250 g of rhizome of swordlike atractylodes, 375 g of patchouli, 500 g of Polygonum cuspidatum, 750 g of verbena, 750 g of reed rhizome, 375 g of lepidium seed, 375 g of pummelo peel, 300 g of artemisia annua and 250 g of licorice. More preferably, the traditional Chinese medicine composition or the traditional Chinese medicine preparation is composed or prepared by 150 g of ephedra, 500 g of Polygonum cuspidatum, 375 g of bitter apricot seed, 750 g of verbena, 750 g of raw gypsum, 750 g of reed rhizome, 750 g of coix seed, 375 g of lepidium seed, 250 g of rhizome of swordlike atractylodes, 375 g of patchouli, 250 g of licorice and 300 g of artemisia annua.

In one embodiment of this aspect, the traditional Chinese medicine composition of the present invention contains a traditional Chinese medicine active extract, the traditional Chinese medicine active extract consists of a first extract and a second extract, and the first extract is an extract obtained by extracting ephedra, bitter apricot seed, raw gypsum, coix seed, rhizome of swordlike atractylodes, patchouli, Polygonum cuspidatum, verbena, reed rhizome, lepidium seed, pummelo peel, and artemisia annua with water or alcohol-water solution, the extraction process is characterized in that the gypsum is firstly decocted, and after extraction, the ephedra, bitter apricot seed, coix seed, rhizome of swordlike atractylodes, Polygonum cuspidatum, verbena, reed rhizome, lepidium seed, and pummelo peel are added to extract, it is filtered, to obtain a first filtrate, the artemisia annua and patchouli are added to a medicinal residue, it is filtered, to obtain a second filtrate, the first filtrate and the second filtrate are combined to obtain the first extract, and the second extract is an extract obtained by extracting the licorice with water or alcohol-water solution, herein the extraction of the first extract and the second extract is performed separately.

In a preferred embodiment of this aspect, the first extract is a water extract or an alcohol-water extract of the following traditional Chinese medicinal materials in parts by weight: 6 parts of ephedra, 15 parts of bitter apricot seed, 30 parts of raw gypsum, 30 parts of coix seed, 10 parts of rhizome of swordlike atractylodes, 15 parts of patchouli, 20 parts of Polygonum cuspidatum, 30 parts of verbena, 30 parts of reed rhizome, 15 parts of lepidium seed, 15 parts of pummelo peel, and 12 parts of artemisia annua; and the second extract is a water extract or an alcohol-water extract of 10 parts of licorice.

It is understood by those skilled in the art that the weight parts of the above traditional Chinese medicinal materials are relative, and the amount of one or more of the traditional Chinese medicinal materials may be adjusted independently and rationally according to a theory of the traditional Chinese medicine. For example, the ephedra may be 4-8 parts, the bitter apricot seed may be 12-18 parts, the raw gypsum may be 24-36 parts, the coix seed may be 24-36 parts, the rhizome of swordlike atractylodes may be 8-12 parts, the patchouli may be 12-18 parts, the Polygonum cuspidatum may be 16-24 parts, the verbena may be 24-36 parts, the reed rhizome may be 24-36 parts, the lepidium seed may be 12-18 parts, the pummelo peel may be 12-18 parts, the artemisia annua may be 9-15 parts, and the licorice may be 8-12 parts. Therefore, all apparent variation forms of this aspect are within the scope of the present invention.

In one embodiment of this aspect, the traditional Chinese medicine active extract of the present invention consists of the first extract and the second extract, herein the first extract may be prepared by a method including the following steps.

(1) the raw gypsum is placed in water which is 3 to 15 times in amount to extract for 10 to 90 minutes, preferably 20 to 40 minutes, and more preferably 30 minutes, then the ephedra, bitter apricot seed, coix seed, rhizome of swordlike atractylodes, Polygonum cuspidatum, verbena, reed rhizome, lepidium seed and pummelo peel are added, and extracted for one or more times with 3 to 15 times of the water in amount, each time is 20 to 90 minutes, it is filtered, and filtrates are combined while being extracted for many times, to obtain a first filtrate, and a medicinal residue is directly used in the next step.

(2), the artemisia annua and patchouli are added to the medicinal residue, 3 to 10 times of the water in amount is added, it is extracted for one or more times, each time is 10 to 80 minutes, it is filtered, and filtrates are combined while being extracted for many times, to obtain a second filtrate.

(3), the first filtrate and the second filtrate are combined, and concentrated, to obtain the first extract.

In another embodiment of this aspect, the traditional Chinese medicine active extract of the present invention consists of the first extract and the second extract, herein the second extract may be prepared by a method including the following steps.

The licorice is extracted for one or more times in 3 to 15 times of water or alcohol-water solution in amount, each time is 20 to 90 minutes, it is filtered, filtrates are combined while being extracted for many times, the filtrate is concentrated, to obtain the second extract.

In yet another embodiment of this aspect, the traditional Chinese medicine active extract of the present invention consists of the first extract and the second extract, herein the first extract is prepared by a method including the following steps.

(1) the raw gypsum is placed in water or alcohol-water solution which is 3 to 15 times in amount to extract for 10 to 90 minutes, preferably 20 to 40 minutes, and more preferably 30 minutes, then the ephedra, bitter apricot seed, coix seed, rhizome of swordlike atractylodes, Polygonum cuspidatum, verbena, reed rhizome, lepidium seed and pummelo peel are added, and extracted for one or more times with 3 to 15 times of the water in amount, each time is 20 to 90 minutes, it is filtered, and filtrates are combined while being extracted for many times, to obtain a first filtrate, and a medicinal residue is directly used in the next step.

(2) the artemisia annua and patchouli are added to the medicinal residue, 3 to 10 times of the water in amount is added, it is extracted for one or more times, each time is 10 to 80 minutes, it is filtered, and filtrates are combined while being extracted for many times, to obtain a second filtrate.

(3) the first filtrate and the second filtrate are combined, and concentrated, to obtain the first extract.

The second extract is prepared by a method including the following steps.

The licorice is extracted for one or more times in 3 to 15 times of water or alcohol-water solution in amount, each time is 20 to 90 minutes, it is filtered, filtrates are combined while being extracted for many times, the filtrate is concentrated, to obtain the second extract.

In a preferred embodiment of this aspect, the traditional Chinese medicine active extract of the present invention consists of the first extract and the second extract, herein the first extract is prepared by a method including the following steps.

(1) the raw gypsum is placed in water or alcohol-water solution which is 10 times in amount to extract for 20 to 40 minutes, and preferably 30 minutes, then the ephedra, bitter apricot seed, coix seed, rhizome of swordlike atractylodes, Polygonum cuspidatum, verbena, reed rhizome, lepidium seed and pummelo peel are added, and extracted for once or twice with 10 times of the water in amount, each time is 1 hour, it is filtered, and filtrates are combined while being extracted for twice, to obtain a first filtrate, and a medicinal residue is directly used in the next step;

(2) the artemisia annua and patchouli are added to the medicinal residue, 6 times of the water in amount is added, it is extracted for once or twice, each time is 20 to 40 minutes, it is filtered, and filtrates are combined while being extracted for twice, to obtain a second filtrate.

(3) the first filtrate and the second filtrate are combined, and concentrated, to obtain the first extract.

In a preferred embodiment of this aspect, the traditional Chinese medicine active extract of the present invention consists of the first extract and the second extract, herein the second extract is prepared by a method including the following steps.

The licorice is extracted for once or twice in 10 times of water or alcohol-water solution in amount, each time is 40 to 80 minutes, preferably 60 minutes, and more preferably 40 minutes, it is filtered, filtrates are combined while being extracted for twice, the filtrate is concentrated, to obtain the second extract.

In a more preferred embodiment of this aspect, the traditional Chinese medicine active extract of the present invention consists of the first extract and the second extract, herein the first extract is prepared by a method including the following steps.

(1) the raw gypsum is placed in water or alcohol-water solution which is 10 times in amount to extract for 20 to 40 minutes, and more preferably 30 minutes, then the ephedra, bitter apricot seed, coix seed, rhizome of swordlike atractylodes, Polygonum cuspidatum, verbena, reed rhizome, lepidium seed and pummelo peel are added, and extracted for once or twice with 10 times of the water in amount, each time is 1 hour, it is filtered, and filtrates are combined while being extracted for twice, to obtain a first filtrate, and a medicinal residue is directly used in the next step.

(2) the artemisia annua and patchouli are added to the medicinal residue, 6 times of the water in amount is added, it is extracted for once or twice, each time is 20 to 40 minutes, it is filtered, and filtrates are combined while being extracted for twice, to obtain a second filtrate.

(3) the first filtrate and the second filtrate are combined, and concentrated, to obtain the first extract.

The second extract is prepared by a method including the following steps.

The licorice is extracted for once or twice in 10 times of water or alcohol-water solution in amount, each time is 40 to 80 minutes, preferably 60 minutes, and more preferably 40 minutes, it is filtered, filtrates are combined while being extracted for twice, the filtrate is concentrated, to obtain the second extract.

In the preparation process of the traditional Chinese medicine active extract of the present invention, the obtained extract solution is preferably filtered, for example, filtered by a 100-300-mesh sieve, and centrifuged and separated by using a method and a device commonly used in the field.

It is known by those skilled in the art that in any implementation schemes of this aspect, the water addition amount, extraction time and number of extraction times are not absolute, namely the use of the corresponding parameter values outside the range and close to the end value of the range may also achieve the purpose of the present invention. Therefore, all apparent variation forms of the above implementation schemes are within the scope of the present invention.

The traditional Chinese medicine composition according to the present invention may also contain a pharmaceutically acceptable auxiliary material in addition to the traditional Chinese medicine active extract of the present invention involved in any forms of the above aspect.

The auxiliary material suitable for the traditional Chinese medicine composition of the present invention includes any auxiliary materials suitable for the traditional Chinese medicine preparation, as long as the auxiliary material does not produce the adverse effect on the quality and performance of the traditional Chinese medicine composition of the present invention. The commonly used auxiliary materials in the traditional Chinese medicine preparation are mainly diluents, wetting agents, disintegrants and the like. The commonly used diluents are mainly sucrose, dextrin, starch, lactose, mannitol, xylitol, bifidose and the like. The commonly used wetting agents are mainly water, different concentrations of ethanol and the like; and a binder is a commonly used polymer binder, and there are many types, such as ethyl cellulose, polyvinyl pyrrolidone, sodium carboxymethyl cellulose, polyethylene glycol alcohol, and sodium alginate. In order to improve the disintegration degree and release degree of the traditional Chinese medicine granules, the suitable disintegrant may be added. The commonly used disintegrant is a microcrystalline cellulose, a sodium carboxymethyl starch and the like. Those skilled in the art may select and determine the suitable auxiliary material and the content thereof in the traditional Chinese medicine composition of the present invention according to the content disclosed in the description.

The traditional Chinese medicine composition of the present invention may also contain suitable additives, and these additives are well-known in the field, such as an emulsifier, a fragrance, a solubilizer, an anti-caking agent, an antifoaming agent, a binder, a buffering agent, a pH regulator, a propellant, a chelating agent, and a preservative.

The traditional Chinese medicine composition according to the present invention may be prepared into an appropriate dosage form according to clinical needs, such as granules, powder, granules, pills, tablets, lozenges, capsules or microcapsules. Preparation methods for these dosage forms are well-known in the field.

According to another aspect of the present invention, a method for preparing the traditional Chinese medicine composition of the present invention is provided, and the method includes the following steps.

(1) the raw gypsum is placed in 3 to 15 times of water or alcohol-water solution in amount to extract for 10 to 90 minutes, preferably 20 to 40 minutes, and more preferably 30 minutes, then the ephedra, bitter apricot seed, coix seed, rhizome of swordlike atractylodes, Polygonum cuspidatum, verbena, reed rhizome, lepidium seed and pummelo peel are added, and extracted for one or more times with 3 to 15 times of the water in amount, each time is 20 to 90 minutes, it is filtered, and filtrates are combined while being extracted for many times, to obtain a first filtrate, and a medicinal residue is directly used in the next step.

(2) the artemisia annua and patchouli are added to the medicinal residue, it is extracted for one or more times with 3 to 10 times of the water in amount, each time is 10 to 80 minutes, it is filtered, filtrates are combined while be extracted for many times, to obtain a second filtrate.

(3) the first filtrate and the second filtrate are combined, and concentrated, to obtain first concentrated solution.

(4) the licorice is placed in 3 to 15 times of the water or alcohol-water solution in amount to extract for one or more times, each time is 20 to 90 minutes, it is filtered, and filtrates are combined while being extracted for many times, the filtrate is concentrated, to obtain second concentrated solution.

(5) the first concentrated solution and the second concentrated solution are combined, and further concentrated to a relative density of about 1.02-1.05 (60° C.), to obtain a final concentrate.

(6) the obtained final concentrate is spray-dried, to obtain medicinal powder.

(7) an appropriate amount of a pharmaceutically acceptable auxiliary material is added to the obtained medicinal powder, and the traditional Chinese medicine composition is prepared.

In a preferred embodiment of this aspect, the preparation method includes the following steps.

(1) the raw gypsum is placed in 10 times of water or alcohol-water solution in amount to extract for 20 to 40 minutes, and more preferably 30 minutes, then the ephedra, bitter apricot seed, coix seed, rhizome of swordlike atractylodes, Polygonum cuspidatum, verbena, reed rhizome, lepidium seed and pummelo peel are added, and extracted for once or twice with 10 times of the water in amount, each time is 1 hour, it is filtered, and filtrates are combined while being extracted for twice, to obtain a first filtrate, and a medicinal residue is directly used in the next step.

(2) the artemisia annua and patchouli are added to the medicinal residue, it is extracted for once or twice with 3 to 10 times of the water in amount, each time is 10 to 80 minutes, it is filtered, filtrates are combined while be extracted for twice, to obtain a second filtrate.

(3) the first filtrate and the second filtrate are combined, and concentrated, to obtain first concentrated solution.

(4) the licorice is placed in 3 to 15 times of the water or alcohol-water solution in amount to extract for once or twice, each time is 20 to 90 minutes, it is filtered, and filtrates are combined while being extracted for twice, the filtrate is concentrated, to obtain second concentrated solution.

(5) the first concentrated solution and the second concentrated solution are combined, and further concentrated to a relative density of about 1.02-1.05 (60° C.), to obtain a final concentrate.

(6) the obtained final concentrate is spray-dried, to obtain medicinal powder.

(7) an appropriate amount of a pharmaceutically acceptable auxiliary material is added to the obtained medicinal powder, and the traditional Chinese medicine composition is prepared.

It is known by those skilled in the art that, in order to promote the achievement of the purpose of the present invention, one or more steps may be added to the above method steps. For example, before the extraction, the traditional Chinese medicinal material may be soaked for a period of time, or the traditional Chinese medicinal material may be physically processed to facilitate the extraction of an active substance. All such apparent variation forms are within the scope of the present invention.

For example, it is known by those skilled in the art that, on the basis of the content disclosed in the description, the corresponding traditional Chinese medicinal materials may be used alone or their mixtures may be used by conventional pulverization, leaching and separation methods in the field, such as immersion, diafiltration, liquid-liquid extraction, water extraction and alcohol precipitation, alcohol extraction and water precipitation, and dialysis, the suitable traditional Chinese medicine active extract in the present invention is prepared. One or more active extracts in the traditional Chinese medicines used in the present invention may also be purchased by a commercial channel, and then combined with extracts of other traditional Chinese medicines, to obtain the traditional Chinese medicine active extract of the present invention. These variation forms are all within the scope of the present invention.

Therefore, according to the above methods and the methods exemplified in the following embodiments, those skilled in the art may easily prepare the traditional Chinese medicine active extract of the present invention, and may further prepare it into a required solid dosage form.

The traditional Chinese medicine composition of the present invention has functions of relieving external appearance and clearing heat, removing dampness and detoxifying, mainly treating dampness-toxin stagnation of the lung, and may be used to treat the diseases caused by the coronaviruses, such as COVID-19. It is proved by test and clinical treatment results that the traditional Chinese medicine composition of the present invention may improve the ability of the patient immune system to fight against the coronaviruses, one or more symptoms of the patient are alleviated or eliminated, the mild disease is not converted into the severe disease or the probability of the conversion is reduced, the severe disease is converted into the common-type or mild-type symptom or the probability of the conversion is increased, the recovery time of the patient is shortened, the course of the disease is shortened, and the nucleic acid turns to negative faster. The traditional Chinese medicine composition of the present invention has remarkable curative effect on the early common-type symptoms and mild-type and common-type patients of COVID-19, and may apparently relieve the symptoms such as fever, cough, wheezing and fatigue, and CT diagnosis also shows that it is significantly improved after the treatment. The control observation of 120 cases in a provincial hospital of integrated traditional Chinese and Western medicine and the treatment results of the patients with COVID-19 in Jiangxia Fangcang Hospital show that the traditional Chinese medicine composition of the present invention has the significant effect on improving the symptoms of COVID-19 (including fever reduction, and treatment of cough, wheezing and fatigue).

Therefore, according to a further aspect of the present invention, the present invention relates to a use of the traditional Chinese medicinal active extract or the traditional Chinese medicine composition of the present invention in preparation of a drug for treating a coronavirus disease (especially COVID-19).

In a variation form of this aspect, the present invention provides a method for treating a patient with a coronavirus disease (for example, COVID-19) using the traditional Chinese medicine composition of the present invention, the method includes administering a therapeutically effective dose of the traditional Chinese medicine composition of the present invention to the patient with the coronavirus disease (for example, COVID-19).

In another variation form of this aspect, the present invention provides a traditional Chinese medicine composition for treating a patient with a coronavirus disease (for example, COVID-19), and the traditional Chinese medicine composition contains the traditional Chinese medicine active extract of the present invention.

The pharmaceutical composition of the present invention may be administered by any suitable modes and in any suitable forms commonly used in the field. For example, the pharmaceutical composition of the present invention may be administered by a mode selected from the followings: oral administration, spray inhalation, nasal administration, and parenteral administration such as intravenous and intramuscular administration, herein the oral administration, intramuscular injection or intravenous injection is preferred.

The traditional Chinese medicine composition of the present invention may be prepared into a unit dosage form for administration of the patent. The dosage form for administration may be a liquid dosage form or a solid dosage form. The liquid dosage form may be solutions, colloids, emulsions, or suspensions, and the like. The solid dosage form may be, for example, tablets, powder, suppositories, granules or capsules. Other dosage forms include aerosols, patches or liniments and the like.

A term “unit dosage form” means a physically discrete unit suitable as a unit dosage for a human subject and other mammals, and each unit contains a predetermined quantity of the traditional Chinese medicine active extract of the present invention calculated to produce the desired therapeutic effect and the suitable pharmaceutical excipient. Usually, the traditional Chinese medicine composition of the present invention may be used orally, for example, twice a day, 5-30 grams each time, such as 10-20 grams, preferably 10 grams of the granules of the present invention or a corresponding amount of other forms of the pharmaceutical composition of the present invention. The specific dosage administered depends upon factors such as the weight of the patient being treated, the nature and severity of the disease, the mode of administration of the drug, and the period or interval of administration. For some patients with special circumstances, specific administration should be prescribed by a doctor.

In addition, the traditional Chinese medicine composition of the present invention may be used in combination with other medicines known in the field that may be used for the treatment of the coronavirus diseases. Those skilled in the art may imagine and determine anti-coronavirus drugs that may be used in combination with the traditional Chinese medicine composition of the present invention and may not produce the adverse effects.

Test Example

The beneficial effects of the traditional Chinese medicine preparation of the present invention are further described below by test examples, these test examples include the clinical curative effect observation of the traditional Chinese medicine of the present invention (hereinafter referred to as decoction for dispersing lung qi and detoxicating).

### Test Example 1: Preparation of Clinical Trial Drug (Traditional Chinese Medicine Decoction) of Present Invention

Formula: 150 g of ephedra, 500 g of Polygonum cuspidatum, 380 g of bitter apricot seed, 740 g of verbena, 750 g of raw gypsum (decocted firstly), 750 g of reed rhizome, 750 g of coix seed, 380 g of lepidium seed, 250 g of rhizome of swordlike atractylodes, 380 g of pummelo peel, 380 g of patchouli (decocted later), 250 g of licorice, and 300 g of artemisia annua (decocted later).

The ephedra, Polygonum cuspidatum, bitter apricot seed, verbena, raw gypsum, reed rhizome, coix seed, lepidium seed, rhizome of swordlike atractylodes, pummelo peel, patchouli, licorice, and artemisia annua medicinal materials are taken, added with water and decocted for twice, for the first time, water which is 8 times greater than the weight of the above medicinal materials in amount is added, and the decocting time is 30 minutes; for the second time, water which is 6 times greater than the weight of the above medicinal materials in amount is added, and the decocting time is 30 minutes; the above decoctions are combined, and filtered, and filtrates are combined; and the above two times of water decoctions are combined, to obtain the clinical trial drug.

### Test Example 2: Summary of Clinical Research of Traditional Chinese Medicine of Present Invention

By prospective randomized controlled trials and retrospective analysis, the clinical efficacy of the traditional Chinese medicine of the present invention in the treatment of mild-type, common-type and severe-type cases of COVID-19 is evaluated, and it is proved that the traditional Chinese medicine of the present invention is an effective prescription for the treatment of COVID-19, and has the good safety.

1.1 Randomized Controlled Trial of Traditional Chinese Medicine of Present Invention for Treating COVID-19 (Mild-Type and Common-Type)

Included in Jiangxia Fangcang Hospital using the traditional Chinese medicine decoction of the present invention, there are 236 cases of patients in a test group, the average length of hospital stay of the subjects in the test group is about 14.80±5.08 days, and the traditional Chinese medicine decoction of the present invention may improve the clinical symptoms of the patients and improve the effect. During an observation period of the clinical treatment, a serious adverse event such as exacerbation is not found. Subgroup analysis shows that the addition of antiviral, antibiotics, hormones and other drugs on the basis of the traditional Chinese medicine decoction does not increase the efficacy, so it is proved that the traditional Chinese medicine (decoction for dispersing lung qi and detoxicating) may be used alone to treat the mild-type and common-type patients with COVID-19.

1.2 Randomized Controlled Clinical Trial of Traditional Chinese Medicine of Present Invention for Treating COVID-19 (Common-Type)

In this research, 68 cases of patients are included in 3 designated hospitals in Henan, herein there are 35 cases in a traditional Chinese medicine group of the present invention, and 33 cases in a control group. The average number of days that the nucleic acid turns to negative in the traditional Chinese medicine group of the present invention is 9.57±3.03 days, and the average number of days that the subjects in the blank control group turns to negative for the nucleic acid is 13.09±5.24 days, the difference between the groups is statistically significant (P<0.05). The curative effect of the traditional Chinese medicine group of the present invention is better than that of the blank control group. The average length of hospital stay in the traditional Chinese medicine group of the present invention is 14.00±6.45 days, and the average length of hospital stay in the blank control group is 16.67±5.57 days. The difference between the groups is statistically significant (P<0.05). The curative effect of the traditional Chinese medicine group of the present invention is better than that of the blank group. There is no serious adverse event reported in the two groups during hospital treatment. Results show that the traditional Chinese medicine of the present invention has the significant curative effect on the treatment of pneumonia caused by COVID-19, and may shorten the time of viral nucleic acid turning to negative and the hospitalization time of the patient.

1.3 Randomized Open Clinical Trial of Traditional Chinese Medicine of Present Invention for Treating COVID-19 (Common-Type)

This research is performed respectively in a certain city hospital of traditional Chinese medicine and a certain provincial hospital of integrated traditional Chinese and western medicine. 71 cases of patients are included in the certain city hospital of traditional Chinese medicine. The CT image improvement rate of a traditional Chinese medicine group of the present invention to a test patient group is tested; and the length of hospital stay in the traditional Chinese medicine group of the present invention is 17.38±7.11 days. 34 cases in the test group are included in the certain provincial hospital of integrated traditional Chinese and Western medicine, research results show that the average length of hospital stay of the patients in the test group is significantly shorter, and the time of the nucleic acid turning to negative is also significantly reduced. There is no serious adverse event such as death occurring in the test group.

1.4 Test Result of Traditional Chinese Medicine of Present Invention for Treating Severe-Type Case of COVID-19

A total of 41 cases in a test group are included in the research, and there are major events during the hospitalization. In the test group, 1 case of the patient dies, and 1 case of the patient generates a multiple organ dysfunction syndrome (MODS); and the average length of hospital stay of the subjects in the test group is 21.42±5.83 days, and the length of hospital stay in the test group is significantly shortened, during a test period, an adverse response related to the traditional Chinese medicine of the present invention is not found.

1.5 Observation Data Analysis for Clinical Safety Case of Traditional Chinese Medicine of Present Invention

Data of 650 cases of patients using the traditional Chinese medicine of the present invention in 7 hospitals including a certain and Henan are summarized and analyzed, herein there are 192 cases of mild-types, 397 cases of common-types, 57 cases of severe-types, and 4 cases of critical-types. 501 cases of the patients are cured and discharged (the cure rate is 77.08%), and 148 cases of the patients are transferred (due to the requirements of epidemic prevention and control policies, a Fangcang hospital is closed or a designated hospital is concentrated in a specialized hospital for further treatment; 5 cases of the severe-type or critical-type patients are transferred to other hospitals), and there is 1 case of death (severe-type patient). During the treatment, a total of 17 adverse events are found, and it is determined to be related to the severe conditions, underlying diseases, drug combination and the like of the subject, and may not be related to the traditional Chinese medicine of the present invention. It is proved that the traditional Chinese medicine of the present invention has the good clinical safety.

2.1 Analysis and Evaluation of Main Research Result

The traditional Chinese medicine of the present invention is a result of summarizing the characteristics and laws of the clinical efficacy of the traditional Chinese medicine on COVID-19.

The clinical trial summary shows that the traditional Chinese medicine of the present invention has the good clinical curative effect on the mild-type, common-type and severe-type patients. Compared with the blank control, the traditional Chinese medicine of the present invention may significantly shorten the virus negative conversion and hospitalization time of the common-type patients; the research data of Fangcang hospital show that, without the use of the western medicines of antiviral, anti-inflammatory and hormone, the single use of the prescription for dispersing lung qi and detoxicating has the significant clinical efficacy, and the addition of the western medicine does not increase the curative effect; and the traditional Chinese medicine of the present invention treats the patients with mild-type and common-type COVID-19, there is no death, aggravation cases, or related adverse responses, it is proved that the prescription may cut off the disease aggravation, and have good clinical safety.

In conclusion, the traditional Chinese medicine of the present invention has the definite curative effect on the treatment of COVID-19, and has the good safety.

### SPECIFIC EMBODIMENTS

In order to further describe the present invention, the following embodiments are provided. These embodiments are intended to illustrate the present invention only, and the scope of the present invention is not limited to the embodiments provided.

The traditional Chinese medicinal materials used in the following embodiments are all purchased from the market and identified to be qualified, the experimental reagents and experimental instruments used are experimental reagents and experimental instruments commonly used in the field, and testing methods used are methods commonly used in the field, unless otherwise specified. For example, the following devices are used in the preparation of the traditional Chinese medicine granules of the present invention in Embodiment 2: a multi-functional extraction tank for a traditional Chinese medicine (T-150, Tianjin Bisheng Pharmaceutical Machinery Co., Ltd.); a combined-type traditional Chinese medicine liquid concentration pot (B-0.5, Tianjin Bisheng Pharmaceutical Machinery Co., Ltd.); a three-dimensional mixer (SYH-50, Changzhou Changhang Drying Instrument Co., Ltd.); a swing granulator (WK-60, Zibo Shike Pharmaceutical Instrument Manufacturing Co., Ltd.); a vacuum drying oven (YZG-1400, Changzhou Yaofei Drying Instrument Technology Co., Ltd.); and a trough mixer (WCH-10, Zibo Shike Pharmaceutical Instrument Manufacturing Co., Ltd.). However, those skilled in the art may know that equivalent devices of these experimental instruments may also be used, as well as other suitable devices depending on the scale of production.

### Embodiment 1

Preparation of Traditional Chinese Medicine Composition (Granules) of Present Invention

Formula Composition:

150 grams of ephedra, 375 grams of bitter apricot seed, 750 grams of raw gypsum, 750 grams of coix seed, 250 grams of rhizome of swordlike atractylodes, 375 grams of patchouli, 750 grams of verbena, 500 grams of Polygonum cuspidatum, 750 grams of reed rhizome, 375 grams of lepidium seed, 375 grams of pummelo peel, 300 grams of artemisia annua and 250 grams of licorice.

Preparation of Traditional Chinese Medicine Active Extract:

For the above thirteen medicinal materials, the licorice is decocted alone, and for the rest of the medicinal materials, the raw gypsum is firstly placed in ten times of water or alcohol-water solution in amount to extract for 30 minutes, and then the rest of the medicinal materials except the artemisia annua and patchouli are added and extracted with ten times of the water in amount for one hour, it is filtered to obtain a filtrate, a medicinal residue is added with the artemisia annua and patchouli, and six times of the water in amount is added to extract for 30 minutes, filtrates are combined, and concentrated below 60° C.; the licorice is extracted for twice with ten times of the water in amount, each time is one hour, and the filtrate is concentrated in an appropriate amount; and concentrated solution is combined to a relative density of about 1.02 (60° C.), and it is spray-dried, to obtain 1100 grams of traditional Chinese medicine active extract powder.

Preparation of Traditional Chinese Medicine Composition:

All of the medicinal powder obtained as above are taken, an appropriate amount of lactose, mannitol and a little microcrystalline cellulose are added, and fully mixed uniformly, 90% ethanol solution is sprayed, it is prepared into granules, and dried at 60° C., to obtain 1500 grams of the granules, namely the traditional Chinese medicine composition (granules) of the present invention.

### Embodiment 2

Preparation of Traditional Chinese Medicine Composition (Granules) of Present Invention

Formula Composition:

240 grams of ephedra, 600 grams of bitter apricot seed, 1200 grams of raw gypsum, 1200 grams of coix seed, 400 grams of rhizome of swordlike atractylodes, 600 grams of patchouli, 1200 grams of verbena, 800 grams of Polygonum cuspidatum, 1200 grams of reed rhizome, 600 grams of lepidium seed, 600 grams of pummelo peel, 480 grams of artemisia annua and 400 grams of licorice.

Preparation of Traditional Chinese Medicine Active Extract:

The indicated amount of decoction pieces (except the licorice) are taken in each batch, and decocted for twice, 10 times of water is added for the first time of the decoction, the raw gypsum is decocted for 30 minutes, then other medicinal materials (except the artemisia annua and patchouli) are decocted for 60 minutes, it is filtered to obtain a first decoction extract, the artemisia annua and patchouli are added along with a first decoction medicinal residue and 6 times of the water is added, the second time of the decoction is performed for 30 minutes, extracted solution is filtered with a 250-mesh filter sieve, and a filtrate is concentrated under a reduced pressure to a specific gravity of 1.03˜1.10 (below 60° C.); and 400 grams of the licorice is taken and decocted alone, 10 times of the water is added for the first time of the decoction, it is decocted for 60 minutes, and 10 times of the water is added for the second time of the decoction, it is decocted for 40 minutes, extracted solution is filtered by the 250-mesh filter sieve, a filtrate is concentrated under a reduced pressure to a specific gravity of 1.03˜1.10 (below 60° C.), and the above concentrated extracts are combined, and spray-dried, to obtain 1200 grams of traditional Chinese medicine active extract powder.

Preparation of Traditional Chinese Medicine Composition:

The dry traditional Chinese medicine active extract powder of the present invention obtained as above is taken, auxiliary materials of the lactose and mannitol (the ratio is 2:1) are added, ethanol with a concentration of 90% is used to prepare granules by a wet method, it is dried (70° C.) for 90 minutes, and granulated, to obtain 1600 grams of the granules, namely the traditional Chinese medicine composition (granules) of the present invention.

### Embodiment 3

Preparation of Traditional Chinese Medicine Composition (Granules) of Present Invention

Formula Composition:

| | | |
|---|---|---|
| Ephedra | 4 | kg |
| Bitter apricot seed | 10 | kg |
| Raw gypsum | 20 | kg |
| Coix seed | 20 | kg |
| Rhizome of swordlike atractylodes | 6.7 | kg |
| Patchouli | 10 | kg |
| Artemisia annua | 8 | kg; |
| Polygonum cuspidatum | 13.3 | kg |
| Verbena | 20 | kg |
| Reed rhizome | 20 | kg |
| Lepidium seed | 10 | kg |
| Pummelo peel | 10 | kg |
| Licorice | 6.7 | kg |

Preparation of Traditional Chinese Medicine Active Extract:

In a multi-functional extraction tank (model DT-3m3, Wenzhou Pharmaceutical Machinery Instrument Factory), the raw gypsum is placed in 10 times of water in amount and decocted for 30 minutes firstly, and then other medicinal materials (except the artemisia annua and patchouli) are added and decocted for 60 minutes, it is filtered to obtain a first decoction extracted solution, the artemisia annua and patchouli are added along with a first decoction residue and 6 times of the water is added, the second decoction is performed for 30 minutes, extracted solution is filtered, and concentrated below 60° C. (a combined-type concentration pot B-11, Tianjin Bidasheng Pharmaceutical Machinery Co., Ltd.) to a relative density of 1.02-1.10 (60° C.); the licorice is decocted alone, 10 times of water is added for the first time of the decoction, and it is decocted for 60 minutes, and 10 times of the water is added for the second time of the decoction, it is decocted for 40 minutes, extracted solution is filtered, and concentrated below 60° C. to a relative density of 1.02-1.10 (60° C.), the above concentrated extracts are combined, and spray-dried (a pilot-scale spray dryer H-Spray 5S, Beijing Hols Biotechnology Co., Ltd.), to obtain 20 kilograms of traditional Chinese medicine active extract dry powder of the present invention.

Preparation of Traditional Chinese Medicine Composition:

The traditional Chinese medicine active extract dry powder of the present invention obtained as above is taken, auxiliary materials (lactose:mannitol=2:1) are added according to dry powder: auxiliary material=2.1˜2.7:1, mixed for 30 min, and put into a trough mixer (model WCH-10, Zibo Shike Pharmaceutical Instrument Manufacturing Co., Ltd.), an appropriate amount of 90% ethanol is added to prepare a soft material, granules are prepared by a wet method, it is dried, and granulated, to obtain 25.8 kilograms of the traditional Chinese medicine composition (granules) of the present invention.

### Embodiment 4

Content determination method for ephedrine hydrochloride and pseudoephedrine hydrochloride in traditional Chinese medicine composition of the present invention

The content of ephedrine hydrochloride and pseudoephedrine hydrochloride in the granules of the present invention is determined by a high performance liquid chromatography (HPLC) method under the following conditions.

Chromatographic condition and system suitability: a high performance liquid chromatograph U3000 (Thermo Fisher Scientific Co., Ltd., USA); chromatographic column: Hlpersil Gold C18 (250×4.6 mm, 5 μm); mobile phase: acetonitrile-0.1% phosphoric acid (containing 0.1% triethylamine) (3:97); detection wavelength: 207 nm; column temperature: 30° C.; flow rate: 1 mL/min; and the theoretical plate number should not be less than 2000 calculated according to a peak of the ephedrine hydrochloride.

Preparation of reference solution: an appropriate amount of an ephedrine hydrochloride reference substance and a pseudoephedrine hydrochloride reference substance are taken, accurately weighed, and methanol is added to prepare mixed solution containing 40 μg per 1 mL respectively.

Preparation of test solution: an appropriate amount of the granules of the present invention prepared according to the method in Embodiment 3 is taken, and grinded finely, 1 g is taken, and accurately weighed, it is placed in a conical flask with a stopper, 25 mL of 70% methanol solution is accurately added, the weight is weighed, and it is ultrasonically treated for 30 minutes, and cooled, the weight is weighed again, 70% methanol is used to make up the lost weight, it is shaken uniformly, and filtered, a subsequent filtrate is taken, to obtain the test solution.

Testing method: 10 μL of the reference solution and the test solution are precisely absorbed respectively, and injected into the liquid chromatograph for measurement.

### Embodiment 5

Content Determination Method of Naringin and Glycyrrhizin in Traditional Chinese Medicine Composition of Present Invention

The content of naringin and glycyrrhizic acid in the granules of the present invention is determined by HPLC under the following conditions.

Chromatographic condition: a high performance liquid chromatograph U3000 (Thermo Fisher Scientific Co., Ltd., USA); chromatographic column: ACQUITY UPLC BEH C18 (2.1×100 mm, 1.7 μm); flow rate: 0.35 mL/min; column temperature: 30° C.; injection volume: 1.0 μL; wavelength: 254 nm/284 nm; mobile phase A: 0.1% formic acid in water, B: acetonitrile, and gradient elution is as follows:

| | | B/0.1% formic |
|---|---|---|
| Time/minute | A/acetonitrile | acid in water |
| 0 | 10% | 90% |
| 5 | 19% | 81% |
| 15 | 32% | 68% |
| 17 | 48% | 52% |
| 20.5 | 48% | 52% |
| 21.5 | 10% | 90% |
| 22 | 10% | 90% |

Preparation of reference substance solution: an appropriate amount of naringin and ammonium glycyrrhizinate reference substance are taken, accurately weighed, and prepared into mixed solution containing 0.8 mg of the naringin and 0.2 mg of the ammonium glycyrrhizin per 1 ml by using methanol, the reference substance solution (weight of glycyrrhizin=weight of ammonium glycyrrhizinate/1.0207) is obtained.

Preparation of test solution: an appropriate amount of the traditional Chinese medicine granules of the present invention prepared according to the method in Embodiment 3 is taken, mixed uniformly, and grinded finely, about 1.0 g is taken, and the weight is weighed, 25 ml of 70% methanol is accurately added, it is ultrasonically treated for 30 minutes, and cooled, the weight is weighed again, and 70% methanol is used to make up the lost weight, it is shaken uniformly, and centrifuged for 10 minutes (the rotation speed is 13000 revolutions per minute), a supernatant is taken, and filtered, and a subsequent filtrate is taken, to obtain the test solution.

Testing method: 10 μL of the reference solution and the test solution are precisely absorbed respectively, and injected into the liquid chromatograph for measurement.

### Embodiment 6

Effect of Traditional Chinese Medicinal Material Extraction Method of Traditional Chinese Medicine Composition of Present Invention on Composition of Extract

In order to determine the effects of single decoction and combined decoction extraction methods of the traditional Chinese medicine composition of the present invention on the content of extract components, the inventors perform the following experimental researches.

Research method: the following seven methods are used for sample preparation: (1) 6 g of ephedra is added to 60 ml of water to reflux-extract for 1 h; (2) 10 g of licorice is added to 100 ml of water to reflux-extract for 1 h; (3) 238 g of the whole prescription medicinal materials are added to 2380 ml of water to reflux-extract for 1 h; (4) the whole prescription without the ephedra is added to 2320 ml of water to reflux-extract for 1 h; (5) the whole prescription without the licorice is added to 2280 ml of water to reflux-extract for 1 h; (6) 238 ml of the whole prescription extracted solution without the licorice is added to 10 ml of single licorice extracted solution; and (7) 228 ml of the whole prescription extracted solution without the ephedra is added to 6 ml of single ephedra extracted solution. After sample preparation, the samples are injected and analyzed to measure the content of the ephedrine hydrochloride, pseudoephedrine hydrochloride and ammonium glycyrrhizinate.

Experimental result: as shown in Table 1, after the ephedra and licorice are decocted together, it is beneficial to the extraction of ephedrine, but whether the licorice is decocted together with the ephedra or with other medicinal materials except the ephedra, the content of the active ingredients may be decreased. Therefore, in the extraction process of the present invention, the licorice is decocted alone, and is not decocted together with other medicinal materials.

TABLE 1
Effects of different extraction methods of ephedra
and licorice on content of related components

| | Ephedrine | Pseudoephedrine | Ammonium |
|---|---|---|---|
| | hydrochloride | hydrochloride | glycyrrhizinate |
| | content | content | content |
| Ephedra single | 0.71% | 0.37% | — |
| decoction | | | |
| Licorice single | — | — | 1.22% |
| decoction | | | |
| Whole prescription | 0.82% | 0.36% | 0.90% |
| combined decoction | | | |
| Whole prescription | 0.82% | 0.39% | 0% |
| without licorice | | | |
| Combined after | 0.79% | 0.38% | 1.08% |
| licorice single | | | |
| decoction | | | |
| Whole prescription | 0% | 0% | 0.91% |
| without ephedra | | | |
| Combined after | 0.71% | 0.35% | 0.89% |
| ephedra single | | | |
| decoction | | | |

### Embodiment 7

Preparation of Traditional Chinese Medicine Tablet According to Present Invention

Formula: 110 g of ephedra, 460 g of Polygonum cuspidatum, 420 g of bitter apricot seed, 790 g of verbena, 790 g of raw gypsum, 710 g of reed rhizome, 790 g of coix seed, 330 g of lepidium seed, 290 g of rhizome of swordlike atractylodes, 330 g of pummelo peel, 420 g of patchouli, 290 g of licorice, and 250 g of artemisia annua.

Preparation Method:

For the above thirteen medicinal materials, the licorice is decocted alone, and for the rest of the medicinal materials, the raw gypsum is firstly placed in 6 times of water in amount to extract for 30 minutes, and then the rest of the medicinal materials except the artemisia annua and patchouli are added and extracted with 12 times of the water in amount for 0.5 hours, it is filtered to obtain a filtrate, a medicinal residue is added with the artemisia annua and patchouli, and 8 times of the water in amount is added to extract for 50 minutes, filtrates are combined, and concentrated below 60° C.; the licorice is extracted for twice with 8 times of the water in amount, each time is one hour, and the filtrate is concentrated in an appropriate amount; and concentrated solution is combined to a relative density of about 1.02 (60° C.), and it is spray-dried, to obtain medicinal powder, all the medicinal powder are taken and added with an appropriate amount of hydroxypropyl methylcellulose, dextrin, and magnesium stearate pharmaceutical excipients, granules are prepared, and pressed into tablets.

### Embodiment 8

Preparation of Traditional Chinese Medicine Capsules According to Present Invention

Formula: 190 g of ephedra, 540 g of Polygonum cuspidatum, 330 g of bitter apricot seed, 710 g of verbena, 710 g of raw gypsum, 790 g of reed rhizome, 710 g of coix seed, 420 g of lepidium seed, 210 g of rhizome of swordlike atractylodes, 420 g of pummelo peel, 330 g of patchouli, 210 g of licorice, and 350 g of artemisia annua.

Preparation Method:

For the above thirteen medicinal materials, the licorice is decocted alone, and for the rest of the medicinal materials, the raw gypsum is firstly placed in 8 times of water in amount to extract for 30 minutes, and then the rest of the medicinal materials except the artemisia annua and patchouli are added and extracted with 8 times of the water in amount for 1 hour, it is filtered to obtain a filtrate, a medicinal residue is added with the artemisia annua and patchouli, and 10 times of the water in amount is added to extract for 50 minutes, filtrates are combined, and concentrated below 60° C.; the licorice is extracted for twice with 6 times of the water in amount, each time is 1 hour, and the filtrate is concentrated in an appropriate amount; and concentrated solution is combined to a relative density of about 1.02 (60° C.), and it is spray-dried, to obtain medicinal powder, all the medicinal powder are taken and added with an appropriate amount of calcium hydrogen phosphate, magnesium oxide, sodium carboxymethyl starch, and talcum powder medicinal excipients, granules are prepared, and pressed into tablets.

### Embodiment 9

Preparation of Traditional Chinese Medicine Dropping Pills According to Present Invention

Formula: 130 g of ephedra, 520 g of Polygonum cuspidatum, 400 g of bitter apricot seed, 730 g of verbena, 770 g of raw gypsum, 730 g of reed rhizome, 770 g of coix seed, 400 g of lepidium seed, 230 g of rhizome of swordlike atractylodes, 350 g of pummelo peel, 400 g of patchouli, 230 g of licorice, and 270 g of artemisia annua.

Preparation Method:

For the above thirteen medicinal materials, the licorice is decocted alone, and for the rest of the medicinal materials, the raw gypsum is firstly placed in 15 times of water in amount to extract for 20 minutes, and then the rest of the medicinal materials except the artemisia annua and patchouli are added and extracted with 12 times of the water in amount for 0.5 hours, it is filtered to obtain a filtrate, a medicinal residue is added with the artemisia annua and patchouli, and 6 times of the water in amount is added to extract for 30 minutes, filtrates are combined, and concentrated below 60° C.; the licorice is extracted for twice with 8 times of the water in amount, each time is 0.5 hours, and the filtrate is concentrated in an appropriate amount; and concentrated solution is combined to a relative density of about 1.02 (60° C.), and it is spray-dried, to obtain medicinal powder, all the medicinal powder are taken and added with an appropriate amount of stearic acid, PEG4000, PEG6000 pharmaceutical excipients, and it is dropped into the dropping pills.

### Embodiment 10

Preparation of Traditional Chinese Medicine Pills According to Present Invention

Formula: 170 g of ephedra, 480 g of Polygonum cuspidatum, 350 g of bitter apricot seed, 770 g of verbena, 730 g of raw gypsum, 770 g of reed rhizome, 730 g of coix seed, 350 g of lepidium seed, 270 g of rhizome of swordlike atractylodes, 400 g of pummelo peel, 350 g of patchouli, 270 g of licorice, and 330 g of artemisia annua.

Preparation Method:

For the above thirteen medicinal materials, the licorice is decocted alone, and for the rest of the medicinal materials, the raw gypsum is firstly placed in 12 times of water in amount to extract for 30 minutes, and then the rest of the medicinal materials except the artemisia annua and patchouli are added and extracted with 9 times of the water in amount for 1 hour, it is filtered to obtain a filtrate, a medicinal residue is added with the artemisia annua and patchouli, and 8 times of the water in amount is added to extract for 40 minutes, filtrates are combined, and concentrated below 60° C.; the licorice is extracted fortwice with 10 times of the water in amount, each time is 1 hour, and the filtrate is concentrated in an appropriate amount; and concentrated solution is combined to a relative density of about 1.02 (60° C.), and it is spray-dried, to obtain medicinal powder, all the medicinal powder are taken and added with an appropriate amount of a honey pharmaceutical excipient, and it is dropped into the dropping pills.

### Embodiment 11

Preparation of Traditional Chinese Medicine Granules According to Present Invention

Formula: 150 g of ephedra, 375 g of bitter apricot seed, 750 g of raw gypsum, 750 g of coix seed, 250 g of rhizome of swordlike atractylodes, 375 g of patchouli, 750 g of verbena, 500 g of Polygonum cuspidatum, 750 g of reed rhizome, 375 g of lepidium seed, 375 g of pummelo peel, 300 g of artemisia annua and 250 g of licorice.

Preparation Method:

For the above thirteen medicinal materials, the licorice is decocted alone, and for the rest of the medicinal materials, the raw gypsum is firstly placed in 10 times of water in amount to extract for 30 minutes, and then the rest of the medicinal materials except the artemisia annua and patchouli are added and extracted with 10 times of the water in amount for 1 hour, it is filtered to obtain a filtrate, a medicinal residue is added with the artemisia annua and patchouli, and 6 times of the water in amount is added to extract for 30 minutes, filtrates are combined, and concentrated below 60° C.; the licorice is extracted for twice with 10 times of the water in amount, each time is 1 hour, and the filtrate is concentrated in an appropriate amount; and concentrated solution is combined to a relative density of about 1.02 (60° C.), and it is spray-dried, to obtain 1100 g of medicinal powder, all the medicinal powder are taken and added with an appropriate amount of lactose, mannitol and a little microcrystalline cellulose, it is mixed uniformly, and 90% ethanol solution is sprayed to prepare granules, and it is dried at 60° C., to prepare 1500 g of the granules.

### Embodiment 12

Therapeutic Effect of Traditional Chinese Medicine Granules According to Present Invention on Disease-Syndrome Combination Model of Human Coronavirus Pneumonia Cold-Damp Epidemic Virus Attacking Lungs in Mice

1 Test Material

1.1 Test Drug

The traditional Chinese medicine granules of the present invention have a production date of 20200306 and a specification of 5 g/bag, which is provided by Modern Chinese Medicine Innovation Center.

Usage: twice a day/2 bags at a time.

1.2 Positive Control Drug

Chloroquine phosphate sugar-coated tablet: the batch number is 2002114, the valid date is until January 2022, and it is produced by Sichuan Shenghe Pharmaceutical Co., Ltd. Usage and dosage: 1-2 days: 1.0 g/60 kg/d; 3-7 days: 0.5 g/60 kg/d.

Recombinant human interferon α2b injection (Pseudomonas): the batch number is R0191201, the valid date is until 2021.11.25, the specification is 3 million IU/1 ml, and it is produced by Tianjin Weiming Biopharmaceutical Co., Ltd. Usage and dosage: 10 million IU//d.

1.3 Experimental Animal

220 BALB/c mice, SPF-grade, weight: 10-12 g, half male and half female, 1100112011011024/5

SCXK (Beijing) 2016-0006

1.4 Virus strain and cell

1.4.1 Virus strain: human coronavirus (HCoV-229E), it is provided by Institute of Medical Biotechnology, Chinese Academy of Medical Sciences, passaged in this laboratory, and stored in a −80° C. refrigerator for future use.

1.4.2 Cell line: Human embryonic lung cells (MRC-5) are purchased from Beijing Beina Chuanglian Institute of Biotechnology, passaged in this laboratory, and stored in liquid nitrogen for future use.

2 Method and Result

2.1 Dosage Design and Drug Formulation

Traditional Chinese medicine granules of present invention: the clinical dosage for humans is 20 g/60 kg/d, namely 0.33 g/kg body weight; the dosage for mice is set to 7.34 g/kg/d, 3.67 g/kg/d, and 1.84 g/kg/d respectively during the test, and the three dosages are equivalent to 2 times, equal times and ½ times of the clinical dosage respectively.

2.2 Virus Passage

A 25 cm² culture flask in which a monolayer of MRC-5 cells is already grown is taken, culture solution is poured out, and after a cell surface is washed with cell maintenance solution for 3 times, 200 μl of HCoV-229E virus solution is added, it is placed in an incubator at 37° C. and 5% CO₂ for culture. A cytopathic condition is observed under an inverted microscope every day, it is lasted for 72˜96 h, until 80% of the cells show the apparent cytopathic effect (CPE), then the cell culture flask is placed in a −80° C. low-temperature refrigerator for freezing, and after the virus solution is repeatedly frozen and thawed for 3 times, it is used to detect the virus virulence.

2.3 Virus Titer Determination

A culture plate on which a monolayer of MRC-5 cells is already grown is taken, culture solution is poured out, and after a cell surface is washed with the cell maintenance solution for 3 times, different titers of HCoV-229E virus solution is inoculated by 10-fold dilution, there is a total of 8 dilution degrees of 10⁻¹˜10⁻⁸, 100 μl/well, and 4 replicate wells for each concentration, and a normal cell control is set at the same time. It is cultured in an incubator at 37° C. and 5% CO₂, a cytopathic condition is observed under the inverted microscope every day, and the cytopathic condition of each well is recorded for 72˜96 hours. 50% cytopathic concentration (TCID50) is calculated by Reed-Muench.

2.4 Modeling and Testing

90 Balb/c mice are taken, SPF-grade, half male and half female, and the body weight is 10-12 g. They are randomly divided into a normal control group, a 229E infection group, a cold-damp control group, a disease-syndrome combination model group of epidemic viruses attacking lungs in mice (hereinafter referred to as an epidemic virus attacking lung model group), a chloroquine phosphate positive drug group, and an interferon α2b positive drug group, and high, medium and low dosage groups of the traditional Chinese medicine granules of the present invention, there are 10 mice in each group. Except for the normal control group and the 229E infection group, the other mice are continuously placed in an artificial climate box with a relative humidity of 90±3%, no wind, and a temperature of 4±2° C. every day, and are taken out after 4 hours of stimulation for 7 consecutive days.

Except for the normal control group and the cold-damp control group, the other mice are infected with 100 TCID50HCOV-229E virus droplets nasally at 50 μL/mice after mild anesthesia with ether on the 5-th and 6-th days of cold-damp stimulation. The administration of each administration group starts on the day of the first infection, and each dosage group of the traditional Chinese medicine granules of the present invention and the chloroquine phosphate positive drug group are administered by gavage at 0.2 ml/10 g; the normal control group, the cold-damp control group, the 229E infection group, the epidemic virus attacking lung model group are given with normal saline under the same conditions; the interferon α2b positive drug group is inhaled with the original drug solution by atomization, and each time is 20 min. It is administered once a day for 3 consecutive days. On the 4-th day of the infection, autopsy is performed after being weighed, and the following indicators are observed and detected.

2.4.1 Observation of traditional Chinese medicine syndrome manifestation: the activity, activity level, skin and hair status and stool status of mice in each group are observed daily.

2.4.2 Lung Index and its Inhibition Rate Calculated by Taking and Weighing Lungs

Lung index=[lung wet weight(g)/body weight(g)]×100

Inhibition rate of lung index=(lung index of model control group−lung index of drug administration group)/(lung index of model control group−lung index of normal control group)*100%

2.4.3 Nucleic Acid Detection in Lung Tissue (RT-PCR Method)

Nucleic Acid Lysis Treatment

After the mice are dissected, lung tissues are separately stored in a −80° C. low-temperature refrigerator; the mouse lung tissue is taken out from the −80° C. low-temperature refrigerator, and placed in a clean mortar, a small amount of liquid nitrogen is poured into and grinded it into powder with a pestle, the powder is collected in a 1.5 ml centrifuge tube and 1 ml of TRIzol Reagent is immediately added, the bottom of the tube is flicked, the sample is mixed as soon as possible to resuspend; the centrifuge tube is horizontally placed at a room temperature, and incubated for 20 min; at 4° C. and 12000 rpm, it is centrifuged for 10 min; a clarified supernatant is transferred to a new 1.5 ml centrifuge tube; 0.2 ml of chloroform is added, a tube cap is closed tightly, the centrifuge tube is shaken vigorously for 15 s, and incubated at the room temperature for 2-3 min until the liquid is stratified; at 4° C. and 12000 rpm, it is centrifuged for 15 min; the clarified supernatant is carefully transferred to a new 1.5 ml centrifuge tube, 0.5 ml of isopropanol is added, mixed uniformly, and incubated at the room temperature for 30 min; at 4° C. and 12000 rpm, it is centrifuged for 10 min; the supernatant is discarded, a precipitate is gently washed with 1 ml of 75% ethanol (so that the white precipitate is gently floated); at 4° C. and 7500 rpm, it is centrifuged for 5 min; the supernatant is absorbed, the RNA precipitate is briefly dried for 5-10 min; and the precipitate is dissolved with 20 μl of diethylpyrocarbonate (DEPC) water, and stored in the −80° C. low-temperature refrigerator.

Nucleic Acid Detection

Control substance nucleic acid treatment: DEPC-H₂O is used as a negative control. The positive control substance is diluted by 10, 100 and 1000-fold gradient.

Reagent preparation: n×18 μl of HCoV-229E nucleic acid fluorescent PCR detection mixed solution, n×1 μl of an internal control substance, and n×1 μl of a RT-PCR enzyme (n is the number of reaction tubes) are taken, shaken and mixed uniformly for several seconds, and it is centrifuged at 3000 rpm for several seconds.

Sampling: 20 μl of the above mixed solution is taken and put it in a polymerase chain reaction (PCR) tube, then 5 μl of sample nucleic acid extraction solution, DEPC-H₂O, and positive control substance are taken and added to the PCR tube respectively, a tube cover is closed, it is centrifuged for several seconds to make all the liquid at the bottom, and a PCR amplification reaction is performed immediately.

Cycle parameters are set as: 45° C.×10 min; 95° C.×15 min; then it is cycled for 40 times according to 95° C.×15 sec→60° C.×60 sec; the single-point fluorescence detection is performed at 60° C., and a reaction system is 25 μl.

2.4.4 Detection of Gastrin (GAS), Motilin (MTL) in Mouse Serum and Inflammatory Factor in Lung Tissue (ELISA Method)

After dissection, mouse plasma is placed at a room temperature for 30 min, and centrifuged at 3000×g for 10 min, and a supernatant is absorbed into a new eppendorf (ep) tube and stored at −20° C. During the detection, it is operated according to kit instructions, and a microplate reader detects each index at 450 nm of the absorbance.

Lung tissue homogenate sample: after weighing the lung tissue from the mouse, the mouse lung tissue is collected and stored at −4° C. After weighing 50 mg of the lung tissue and adding 500 μL of normal saline, the tissue is homogenized with an ultrasonic cell disruptor, and centrifuged at −4° C. at 1000 xg for 10 minutes with a lower-temperature high-speed centrifuge. After the supernatant is absorbed, it is separately packaged, and stored in the −80° C. refrigerator for later use. Repeated freezing and thawing is avoided. During the detection, it is operated according to the kit instructions, and the microplate reader detects each index at 450 nm of the absorbance.

2.4.5 Flow Cytometry Detection of Peripheral Blood T Lymphocyte Subset and B Lymphocyte Proportion in Mice

The centrifuge is pre-cooled at 4° C. Mouse eyes are removed and blood is taken, 3 drops of the blood (about 150 μl) is added to a 15 ml centrifuge tube loaded with 10 ml of 1×PBS, and it is centrifuged at 1600 rpm for 5 min at a room temperature; the supernatant is carefully discarded with a pipette, and 1 ml of red blood cell lysate is added to each tube to resuspend a cell pellet, it is lysed at the room temperature for about 5-10 min until the liquid becomes clear from turbidity, 10 ml of a phosphate buffer (PBS) is added to terminate the lysis, it is centrifuged at 2000 rpm and 4° C. for 5 min, and the supernatant is discarded. The cell pellet is resuspended with 10 ml of PBS, it is centrifuged at 2000 rpm and 4° C. for 5 min, the supernatant is discarded, it is resuspended with 200 μl of blocking solution (PBS containing 5% fetal bovine serum (FBS)), and cell suspension is transferred to a 1.5 ml ep tube, and blocked at 4° C. for 30 min. A flow cytometry antibody is prepared in the blocking solution in the dark as follows: FITC-labeled anti-mouse CD3e, PE-labeled anti-mouse CD19, PerCP-Cy5.5-labeled anti-mouse CD4, and APC-labeled anti-mouse CD8a, the preparation volume of each tube of the cells is: 0.3 μl of each antibody and 50 μl of the blocking solution.

The cell suspension is centrifuged at 2000 rpm and 4° C. for 5 min, and the supernatant is discarded. The flow cytometry antibody is added at 50 μl per tube, it is stained at 4° C. for 30 min in the dark, 1 ml of PBS is added, it is centrifuged at 2000 rpm and 4° C. for 5 min, and the supernatant is discarded. The cells are resuspended with 200 μl of PBS containing 2% FBS, and transferred to a flow cytometry tube, and detected on a machine.

2.5 Test Result

2.5.1 Influence on Traditional Chinese Medicine Syndrome Manifestation

The model mice are placed in the intelligent artificial climate box, on the 4-th day after being stimulated by cold and dampness, they appear to stay together, the activity is decreased, the activity level is reduced, and at the same time, they show irritability, biting and fighting manifestations, wet and tangled fur, and light and sticky stools, it is indicated that the symptoms of the cold-damp syndrome are present at the beginning. On the 5-th day of the cold-damp stimulation, the coronavirus infection is loaded until the 4-th day after the infection. The mice in the model group show a phenomenon of staying together, the activity level is reduced significantly, the tussle manifestation no longer appears, the fur shows dull and dry, and the color of the stool becomes dark and dry. The characteristics accord with the manifestations of the traditional Chinese medicine syndrome, the disease-syndrome combination model of the human coronavirus pneumonia cold-damp epidemic virus attacking lungs in mice is formed. Compared with the epidemic virus attacking lung model group, the three dosage groups of the traditional Chinese medicine granules of the present invention have the significantly increased activity and reaction ability of mice, and the fur and stool states are improved to a certain extent.

2.5.2 Effect on Lung Index of Mice

TABLE 2
Therapeutic effect of traditional Chinese medicine of present invention
on disease-syndrome combination model of human coronavirus pneumonia
cold-damp epidemic virus attacking lungs in mice

| | | | Number of | Lung index | |
|---|---|---|---|---|---|
| | | Dosage | animals | (Lung weight/100 | Inhibition |
| Group | | (g/kg/d) | (piece) | g body weight) | rate % |
| Normal control group | | — | 10 | 0.67 ± 0.04 | — |
| Cold-damp control group | | — | 10 | 0.69 ± 0.03 | — |
| 229E infection group | | — | 10 | 0.83 ± 0.05^{##} | — |
| Epidemic virus attacking | | — | 10 | 0.87 ± 0.07^{##} | — |
| lung model group | | | | | |
| Chloroquine phosphate | | Id: 0.18 g/kg/d | 7 | 0.85 ± 0.11 | 9.82 |
| group | | 2-3: 0.09 g/kg/d | | | |
| Interferon α2b group | | 3 million IU, | 10 | 0.79 ± 0.11 | 40.38 |
| | | 20 min | | | |
| XFBD | High | 7.34 | 10 | 0.76 ± 0.09** | 54.96 |
| granules | dosage | | | | |
| | Medium | 3.67 | 10 | 0.70 ± 0.06** | 81.08 |
| | dosage | | | | |
| | Low | 1.84 | 10 | 0.75 ± 0.03** | 58.48 |
| | dosage | | | | |

Note:
^{##}p < 0.01 compared with the normal control group; and
**p < 0.01 compared with the epidemic virus attacking lung model group.

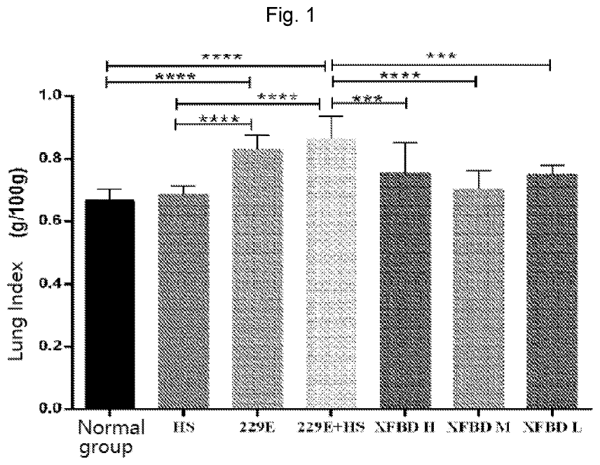

The results in Table 2 and FIG. 1 show that: the lung index of the mice in the epidemic virus attacking lung model group is increased significantly, and there is a significant difference compared with the normal control group (P<0.01); all the high, medium and low dosages of the traditional Chinese medicine granules of the present invention may significantly reduce the lung index of the mice, there is a significant difference compared with the epidemic virus attacking lung model group (P<0.01), and the inhibition rates of the lung index are 54.96%, 81.08% and 58.48% respectively. It is indicated that the traditional Chinese medicine granules of the present invention have the apparent therapeutic effect on the disease-syndrome combination model of human coronavirus pneumonia cold-damp epidemic virus attacking lungs in the mice.

2.5.3 Effect on Mouse Serum MTL and GAS

TABLE 3
Therapeutic effect of traditional Chinese medicine of present invention
on disease-syndrome combination model of human coronavirus pneumonia
cold-damp epidemic virus attacking lungs in mice

| Group | | Dosage | Factor content in serum (pg/ml) | |
|---|---|---|---|---|
| (6/group) | | (g/kg/d) | GAS | MTL |
| Normal control group | | — | 17.89 ± 2.18 | 112.86 ± 22.84 |
| Cold-damp control group | | — | 8.71 ± 1.26^{##} | 155.03 ± 13.16^{##} |
| 229E infection group | | — | 19.31 ± 2.67 | 105.48 ± 10.22 |
| Epidemic virus attacking | | — | 10.48 ± 1.40^{##} | 157.20 ± 10.07^{##} |
| lung model group | | | | |
| Chloroquine phosphate | | Id: 0.18 g/kg/d, | 8.75 ± 1.41 | 105.69 ± 16.85** |
| group | | 2~3: 0.09 g/kg/d | | |
| Interferon α2b group | | 300 million IU, | 8.94 ± 1.07 | 115.43 ± 7.90** |
| | | 20 min | | |
| Traditional | High | 7.34 | 23.42 ± 3.25** | 126.83 ± 20.41** |
| Chinese | dosage | | | |
| medicine | Medium | 3.67 | 21.15 ± 1.58** | 139.59 ± 16.33* |
| granules of | dosage | | | |
| present | Low | 1.84 | 24.01 ± 10.09** | 149.01 ± 22.28 |
| invention | dosage | | | |

Note:
Compared with the normal control group,
^{##}P < 0.01; and compared with the epidemic virus attacking lung model group,
**P < 0.01.

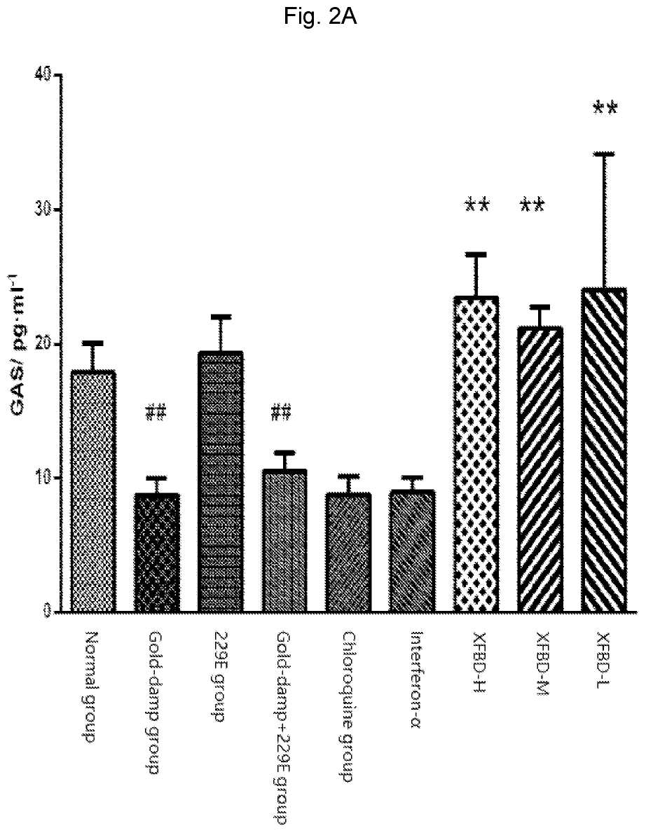

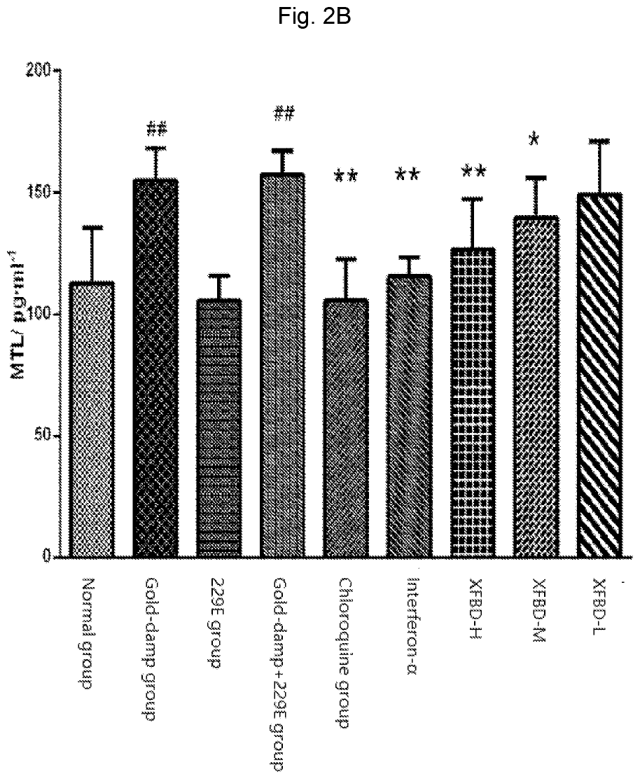

The results of Table 3, FIGS. 2A and 2B show that: the GAS content in the serum of the mice of the epidemic virus attacking lung model group is significantly reduced, the MTL content is significantly increased, and there is a significant difference compared with the normal control group (P<0.01); the three dosage groups of the traditional Chinese medicine granules of the present invention may significantly increase the GAS content, and the high and medium dosage groups may significantly reduce the MTL content, and there are significant differences compared with the epidemic virus attacking lung model group (P<0.05, P<0.01).

2.5.4 Effect on Viral Load in Mouse Lung Tissue

TABLE 4
Therapeutic effect of traditional Chinese medicine of present invention
on disease-syndrome combination model of human coronavirus pneumonia
cold-damp epidemic virus attacking lungs in mice

| | | | Number of | |
|---|---|---|---|---|
| | | Dosage | animals | |
| Group | | (g/kg/d) | (piece) | Viral load |
| Normal control group | | — | 10 | Negative |
| Cold-damp control group | | — | 10 | Negative |
| 229E infection group | | — | 10 | 135713.63 ± 81704.30 |
| Epidemic virus attacking | | — | 10 | 121477.69 ± 35035.81^{##} |
| lung model group | | | | |
| | | Id: 0.18 g/kg/d | 7 | 56027.66 ± 31638.92** |
| Chloroquine phosphate | | 2~3 d: | | |
| group | | 0.09g/kg/d | | |
| Interferon α2b group | | 300 million IU, | 10 | 63758.38 ± 17530.19** |
| | | 20 min | | |
| | High | 7.34 | 10 | 76053.48 ± 30673.93* |
| | dosage | | | |
| Traditional | Medium | 3.67 | 10 | 32514.58 ± 37364.92** |
| Chinese | dosage | | | |
| medicine | | | | |
| granules of | | | | |
| present | | | | |
| invention | | | | |
| | Low | 1.84 | 10 | 61379.29 ± 24918.82** |
| | dosage | | | |

Note:
Compared with the normal control group,
^{##}P < 0.01; compared with the epidemic virus attacking lung model group,
**P < 0.01.

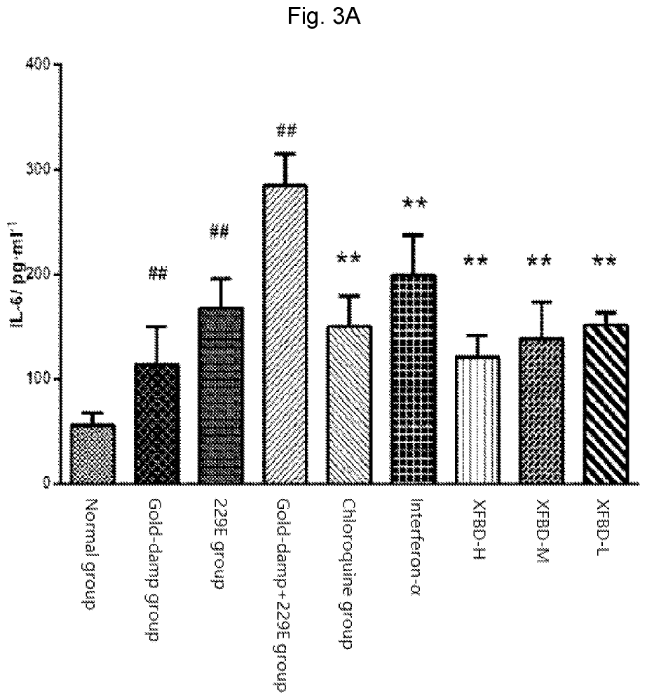

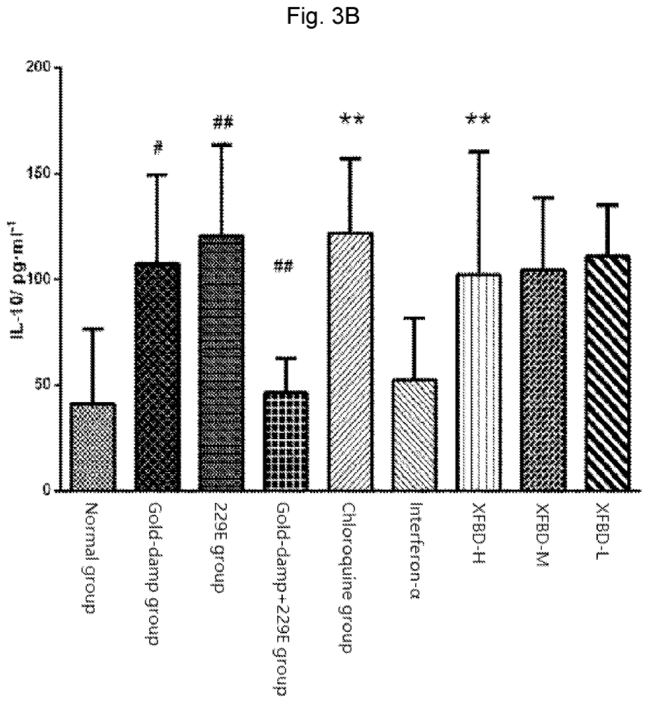

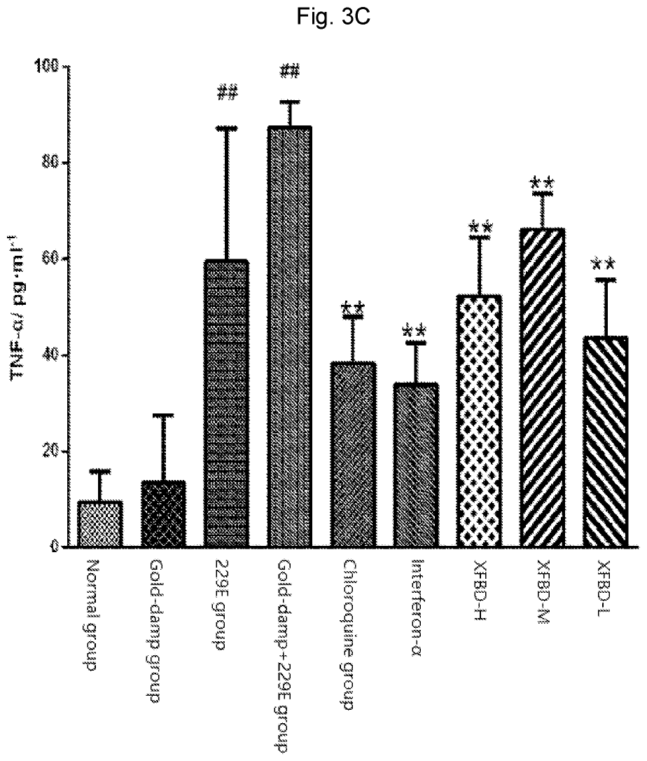

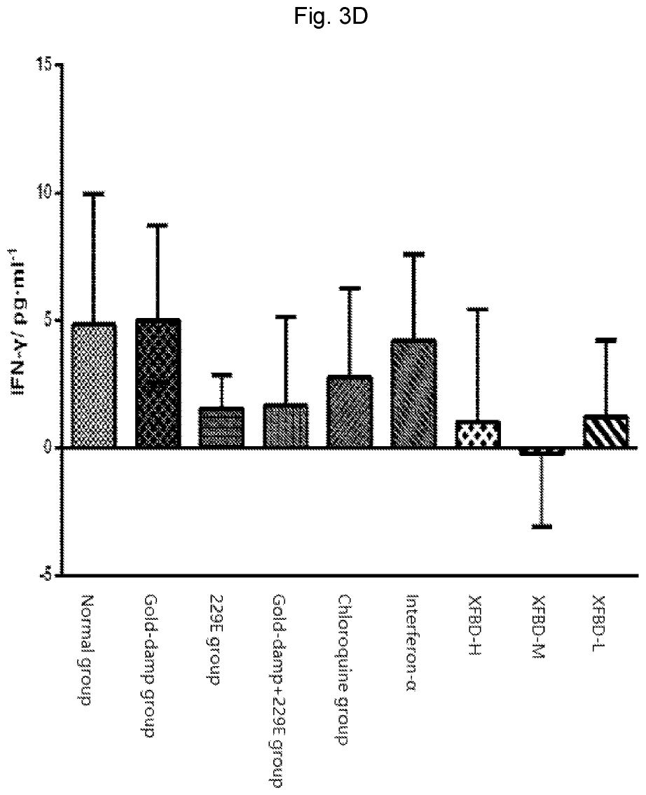

The results of Table 4, FIGS. 3A and 3B show that: there is no HCoV-229E nucleic acid expression in the lung tissues of the normal control group and the cold-damp control group; there is significant nucleic acid expression in the mouse lung tissue of the epidemic virus attacking lung model group; the three dosage groups of the traditional Chinese medicine granules of the present invention may significantly reduce the expression of viral nucleic acids in the lung tissue.

2.5.5 Effect on Content of Inflammatory Factors in Mouse Lung Tissue

TABLE 5
Therapeutic effect of traditional Chinese medicine of present invention on disease-syndrome combination
model of human coronavirus pneumonia cold-damp epidemic virus attacking lungs in mice

| | | Dosage | Inflammatory factor content in lung tissue (pg/ml) | | | |
|---|---|---|---|---|---|---|
| Group (6/group) | | (g/kg/d) | IL-6 | IL-10 | TNF-α | INF-γ |
| Normal control group | | — | 56.12 ± 11.56 | 41.23 ± 35.62 | 9.48 ± 6.34 | 4.85 ± 5.10 |
| Cold-damp control group | | — | 113.90 ± 35.72^{##} | 107.17 ± 42.27^{#} | 13.53 ± 13.95 | 4.99 ± 3.75 |
| 229E infection group | | — | 167.66 ± 28.04^{##} | 20.36 ± 43.41^{##} | 59.54±27.60^{##} | 1.54 ± 1.31 |
| Epidemic virus attacking | | — | 284.75 ± 30.38^{##} | 121.96 ± 35.13^{##} | 87.35 ± 5.41^{##} | 1.65 ± 3.48 |
| lung model group | | | | | | |
| Chloroquine phosphate | | Id: 0.18 g/kg/d | 149.98 ± 29.37** | 52.54 ± 29.40** | 38.33 ± 9.62** | 2.77 ± 3.48 |
| group | | 2~3 d: 0.09 g/kg/d | | | | |
| Interferon α2b group | | 300 million IU, 20 min | 199.07 ± 38.37** | 102.25 ± 58.14 | 33.86 ± 8.72** | 4.20 ± 3.39 |
| Traditional Chinese | High dosage | 7.34 | 120.88 ± 20.96** | 46.701 ± 6.05** | 52.25 ± 12.29** | 1.00 ± 4.42 |
| medicine granules | Medium dosage | 3.67 | 138.41 ± 34.98** | 104.57 ± 34.12 | 66.20 ± 7.39** | −0.23 ± 2.85 |
| of present invention | Low dosage | 1.84 | 151.49 ± 11.98** | 111.06 ± 24.11 | 43.59 ± 12.07** | 1.22 ± 3.00 |

Note:
Compared with the normal control group, ^{#}P < 0.05, and ^{##}P < 0.01; compared with the epidemic virus attacking lung model group, **P < 0.01.

The results of Table 5 show that the contents of the inflammatory factors IL-6, IL-10 and TNF-a in the lung tissue of the mice in the epidemic virus attacking lung model group are significantly increased, and there is a significant difference compared with the normal control group (P<0.01); and the three dosage groups of the traditional Chinese medicine granules of the present invention may significantly reduce the contents of IL-6 and TNF-α, the high dosage group may significantly reduce the content of IL-10, and there is a significant difference compared with the epidemic virus attacking lung model group (P<0.01).

2.5.6 Effect on Percentage of Lymphocytes in Peripheral Blood of Mice

TABLE 6
Therapeutic effect of traditional Chinese medicine of present invention on disease-syndrome combination
model of human coronavirus pneumonia cold-damp epidemic virus attacking lungs in mice

| | | | Percentage of immune cells in peripheral blood (%) | | | |
|---|---|---|---|---|---|---|
| | | Dosage | | | CD4⁺/ | |
| Group (6/group) | | (g/kg/d) | CD4⁺T cell | CD8⁺T cell | CD8⁺ | B cell |
| Normal control group | | — | 42.00 ± 9.87 | 24.38 ± 2.19 | 1.75 ± 0.56 | 23.05 ± 3.25 |
| Cold-damp control group | | — | 30.72 ± 13.64 | 15.20 ± 5.29^{##} | 1.98 ± 0.34 | 18.10 ± 8.61 |
| 229E infection group | | — | 29.70 ± 11.96 | 7.47 ± 5.11^{##} | 4.92 ± 2.51^{#} | 7.64 ± 7.89^{##} |
| Epidemic virus attacking | | — | 26.56 ± 10.71^{#} | 7.46 ± 5.92^{##} | 5.03 ± 2.99^{#} | 8.50 ± 8.80^{##} |
| lung model group | | | | | | |
| Chloroquine phosphate | | Id: 0.18 g/kg/d | 32.47 ± 4.65 | 20.03 ± 3.68** | 1.65 ± 0.23* | 15.72 ± 2.24 |
| group | | 2~3 d: 0.09 g/kg/d | | | | |
| Interferon α2b group | | 300 million IU, 20 min | 35.17 ± 4.24 | 19.68 ± 4.53** | 1.92 ± 0.63* | 6.40 ± 0.98 |
| Traditional Chinese | High dosage | 7.34 | 40.18 ± 5.13* | 25.03 ± 3.31** | 1.66 ± 0.49* | 18.30 ± 0.97* |
| medicine granules of | Medium dosage | 3.67 | 32.75 ± 3.85 | 25.07 ± 3.82** | 1.35 ± 0.36* | 15.10 ± 1.67 |
| present invention | Low dosage | 1.84 | 36.05 ± 1.16 | 25.97 ± 2.98** | 1.41 ± 0.23* | 16.28 ± 0.84 |

Note:
Compared with the normal control group, ^{##}P < 0.01; and compared with the epidemic virus attacking lung model group, *P < 0.05, and **P < 0.01.

The results in Table 6 show that the percentages of the immune cells of CD4+ T cells, CD8+T cells, and B cells in the peripheral blood of the mice in the epidemic virus attacking lung model group are significantly decreased, and there is a significant difference compared with the normal control group (P<0.05, P<0.01); the high dosage group of the traditional Chinese medicine granules of the present invention may significantly increase the percentages of CD4+ T cells, CD8+T cells and B cells, and the medium dosage and low dosage groups may significantly increase the percentage of CD8+T cells, the three dosage groups may significantly increase the CD4+/CD8+ value, and there is a significantly difference compared with the epidemic virus attacking lung model group (P<0.05, P<0.01).

### IN CONCLUSION

The disease-syndrome combination model of human coronavirus pneumonia cold-damp epidemic virus attacking lungs in the mice is used, and by observing the indicators such as the appearance and behavioral manifestations of the traditional Chinese medicine syndromes in the mice, the changes of gastrointestinal hormones in the serum; the lung index, the expression of the viral nucleic acids in the lung tissue, and the inflammatory cytokine content; and the peripheral blood immune cell percentage and the lung tissue pathological detection, the therapeutic effect of the traditional Chinese medicine granules of the present invention on the disease-syndrome combination model of human coronavirus pneumonia cold-damp epidemic virus attacking lungs in the mice is evaluated.

After the mice are modeled, three dosages of 7.34 g/kg/d, 3.67 g/kg/d and 1.84 g/kg/d (equivalent to clinically 2 times, equal times and ½ times respectively) of the Chinese medicine granules of the present invention are given by gavage, once a day for 3 consecutive days. The results show that:

1. The three dosages of the traditional Chinese medicine granules of the present invention may significantly reduce the lung index of the mice, and the lung index inhibition rates are 54.96%, 81.08%, and 58.48% respectively.

2. The three dosages of the traditional Chinese medicine granules of the present invention may significantly reduce the expression of viral nucleic acids in the lung tissue of the mice.

3. The three dosages of the traditional Chinese medicine granules of the present invention may significantly increase the GAS content in the mice serum, and high and medium dosages may significantly reduce the MTL content in the serum.

4. The high dosage group of the traditional Chinese medicine granules of the present invention may significantly increase the percentage of CD4+ T cells, CD8+ T cells and B cells, the medium dosage and low dosage groups may significantly increase the percentage of CD8+T cells, and the three dosages may significantly increase the CD4+/CD8+ value.

5. The three dosage groups of the traditional Chinese medicine granules of the present invention may significantly reduce the content of IL-6 and TNF-α in the lung tissue of the mice; the high dosage group significantly reduces the content of IL-10 in the lung tissue of the mice; and there is no significant effect on the content of IFN-γ in the lung tissue.

The above research results show that the traditional Chinese medicine granules of the present invention have the apparent therapeutic effects on the disease-syndrome combination model of human coronavirus pneumonia cold-damp epidemic virus attacking lungs in the mice, and provide a laboratory basis for clinical medication of COVID-19.

What is claimed is:

1. A traditional Chinese medicine, wherein the traditional Chinese medicine is prepared by the following raw material medicines in parts by weight: 110˜190 parts of ephedra, 460˜540 parts of Polygonum cuspidatum, 330˜420 parts of bitter apricot seed, 710˜790 parts of verbena, 710˜790 parts of raw gypsum, 710˜790 parts of reed rhizome, 710˜790 parts of coix seed, 330˜420 parts of lepidium seed, 210˜290 parts of rhizome of swordlike atractylodes, 330˜420 parts of pummelo peel, 330˜420 parts of patchouli, 210˜290 parts of licorice, and 250˜350 parts of artemisia annua.

2. The traditional Chinese medicine according to claim 1, wherein the traditional Chinese medicine is prepared by the following raw material medicines in parts by weight: 130˜170 parts of ephedra, 480˜520 parts of Polygonum cuspidatum, 350˜400 parts of bitter apricot seed, 730˜770 parts of verbena, 730˜770 parts of raw gypsum, 730˜770 parts of reed rhizome, 730˜770 parts of coix seed, 350˜420 parts of lepidium seed, 230˜270 parts of rhizome of swordlike atractylodes, 350˜400 parts of pummelo peel, 350˜400 parts of patchouli, 230˜270 parts of licorice, and 270˜330 parts of artemisia annua.

3. The traditional Chinese medicine according to claim 2, wherein the traditional Chinese medicine preparation is prepared by the following raw material medicines in parts by weight: 150 parts of ephedra, 500 parts of Polygonum cuspidatum, 375 parts of bitter apricot seed, 750 parts of verbena, 750 parts of raw gypsum, 750 parts of reed rhizome, 750 parts of coix seed, 375 parts of lepidium seed, 250 parts of rhizome of swordlike atractylodes, 375 parts of pummelo peel, 375 parts of patchouli, 250 parts of licorice, and 300 parts of artemisia annua.

4. The traditional Chinese medicine active extract according to claim 1, wherein the traditional Chinese medicine active extract consists of a first water extract and a second water extract, the first water extract is a water extract of the ephedra, bitter apricot seed, raw gypsum, coix seed, rhizome of swordlike atractylodes, patchouli, Polygonum cuspidatum, verbena, reed rhizome, lepidium seed, pummelo peel, and artemisia annua, the second water extract is a water extract of the licorice, and the first water extract and the second extract are obtained by extracting separately.

5. The traditional Chinese medicine active extract according to claim 4, wherein the first water extract is prepared by a method comprising the following steps:
(1) placing the raw gypsum in water which is 3 to 15 times in amount to extract for 10 to 90 minutes, then adding the ephedra, bitter apricot seed, coix seed, rhizome of swordlike atractylodes, Polygonum cuspidatum, verbena, reed rhizome, lepidium seed and pummelo peel, and extracting for one or more times with 3 to 15 times of the water in amount, wherein each time is 20 to 90 minutes, filtering, and combining filtrates while being extracted for many times, to obtain a first filtrate, and directly using a medicinal residue in the next step;
(2), adding the artemisia annua and patchouli to the medicinal residue, adding 3 to 10 times of the water in amount, extracting for one or more times, wherein each time is 10 to 80 minutes, filtering, and combining filtrates while being extracted for many times, to obtain a second filtrate; and
(3), combining the first filtrate and the second filtrate, and concentrating, to obtain the first extract.

6. The traditional Chinese medicine active extract according to claim 4, wherein the first water extract is prepared by a method comprising the following steps:
(1), placing the raw gypsum in water solution which is 10 times in amount to extract for 20 to 40 minutes, then adding the ephedra, bitter apricot seed, coix seed, rhizome of swordlike atractylodes, Polygonum cuspidatum, verbena, reed rhizome, lepidium seed and pummelo peel, and extracting for once or twice with 10 times of the water in amount, wherein each time is 1 hour, filtering, and combining filtrates while being extracted for twice, to obtain a first filtrate, and directly using a medicinal residue in the next step;
(2), adding the artemisia annua and patchouli to the medicinal residue, adding 6 times of the water in amount, and extracting for once or twice, wherein each time is 20 to 40 minutes, filtering, and combining filtrates while being extracted for twice, to obtain a second filtrate; and
(3), combining the first filtrate and the second filtrate, and concentrating, to obtain the first extract.

7. The traditional Chinese medicine active extract according to claim 5, wherein the second water extract is prepared by a method comprising the following steps:
placing the licorice in 3 to 15 times of water in amount and extracting for one or more times, wherein each time is 20 to 90 minutes, filtering, combining filtrates while being extracted for many times, concentrating the filtrate, to obtain the second water extract.

8. The traditional Chinese medicine active extract according to claim 5, wherein the second water extract is prepared by a method comprising the following steps:
placing the licorice in 10 times of water in amount and extracting for once or twice, wherein each time is 40 minutes, filtering, combining filtrates while being extracted for twice, concentrating the filtrate, to obtain the second water extract.

9. A traditional Chinese medicine for dispersing lung qi and detoxicating, comprising the traditional Chinese medicine according to claim 1.

10. A drug for treating a coronavirus disease comprising the traditional Chinese medicine according to claim 1.

11. The drug according to claim 10, wherein the coronavirus disease is a novel coronavirus (COVID-19).

12. A method for treating a coronavirus disease in a subject in need thereof, comprising: administering a therapeutically effective amount of the traditional Chinese medicine according to claim 1 to the subject.

13. The method according to claim 12, wherein the coronavirus disease is COVID-19.
